(19) United States
(12) Patent Application Publication
(10) Pub. No.: US 2025/0109433 A1
(43) Pub. Date: Apr. 3, 2025

(54) MACROMOLECULE BLOCKS FOR NANOPORE SEQUENCING

(71) Applicant: Illumina, Inc., San Diego, CA (US)

(72) Inventors: Hassan Zakiruddin Bohra, Singapore (SG); Yin Nah Teo, Singapore (SG); Ramesh Neelakandan, Singapore (SG); Erin Garcia, El Cajon, CA (US); Abdul Sadeer Abd Salam, Singapore (SG); Daniel Hartoyo Lukamto, Singapore (SG); Soumadwip Ghosh, La Jolla, CA (US); Xiangyuan Yang, Singapore (SG); Jeffrey G. Mandell, Rancho Santa Fe, CA (US)

(21) Appl. No.: 18/887,792

(22) Filed: Sep. 17, 2024

Related U.S. Application Data
(60) Provisional application No. 63/584,624, filed on Sep. 22, 2023.

(51) Int. Cl. C12Q 1/6869; C07H 21/02; C07H 21/04; C12Q 1/6853

(52) U.S. Cl. CPC C12Q 1/6869; C07H 21/02; C07H 21/04; C12Q 1/6853

(57) ABSTRACT

In one aspect, the disclosed technology relates to nanopore sequencing with a polynucleotide comprising a plurality of nucleotides, wherein each nucleotide comprises a macromolecular block. In some embodiments, the macromolecular blocks are configured for slowing or halting the polynucleotide translocation through a nanopore. In some embodiments the macromolecular blocks have linear and branched structures.

### INCORPORATION BY REFERENCE TO RELATED APPLICATION

Any and all applications for which a foreign or domestic priority claim is identified in the Application Data Sheet as filed with the present application are hereby incorporated by reference under 37 CFR 1.57.

### BACKGROUND

Some polynucleotide sequencing techniques involve performing a large number of controlled reactions on support surfaces or within predefined reaction chambers. The controlled reactions may then be observed or detected, and subsequent analysis may help identify properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic electrical current. For example, as the polynucleotide traverses through the nanopore, it influences the electrical current through the nanopore. Each passing nucleotide, or series of nucleotides, that passes through the nanopore yields a characteristic blockage current. These characteristic electrical currents of the traversing polynucleotide can be recorded to determine the sequence of the polynucleotide.

### SUMMARY

The readhead of nanopores (e.g., the constriction region of nanopores) usually reports the summation of all bases residing in the readhead concurrently along the sample DNA strand, increasing the challenge of accurate nanopore sequencing due to many permutations of signals arising from different sequences. For example, the MspA pore reads about 4 bases at a time, giving rise to at least 4\hat{ }4=256 different signals that need to be deconvoluted and resolved.

In one aspect, the disclosed technology provides a method of directly sequencing a native strand of nucleic acids modified with various chemical moieties, including macromolecular blocks, linked via chemical/enzymatic means to attenuate translocation, attenuate enzymatic synthesis, or attenuate any signal generated as a consequence of the nucleotide passing through the readhead. In another aspect, the disclosed technology provides a method that instead of directly sequencing the sample DNA, a daughter strand is synthesized using natural or synthetic nucleotides, wherein the resulting daughter strand further features the addition of various chemical moieties, including macromolecular blocks, linked via chemical/enzymatic means to attenuate translocation, attenuate enzymatic synthesis, or attenuate any signal generated as a consequence of the nucleotide passing through the readhead.

In some embodiments, during sequencing of a polynucleotide sequence, a system can “read” elements associated with each nucleotide sequence, as each nucleotide passes through the nanopore readhead to generate an output. The output can be a signal that can be used to identify a particular base. Macromolecular blocks chemically linked to a nucleotide strand or translocating polymer are designed to occupy and interact non-covalently with chemical or amino acid residues within the inner environment of a nanopore, thereby affecting signal generation, and/or the rate of translocation. These interactions include, but are not limited to, electrostatic interactions, ion-dipole interactions, dipole-dipole (e.g. hydrogen bonding) interactions, induced-dipole interactions, hydrophobic interactions, London dispersion forces interactions, Van-der-Waals interactions, hydrophobic interactions, cation-pi, anion-pi, and pi-pi interactions. In some embodiments, the macromolecular block can modulate signal generation, thereby producing a distinguishable signal or signal break. In some embodiments, the macromolecular block can act as a “brake” or “ratchet”—wherein an associated nucleotide translocates only upon application of a voltage past a certain threshold, or a current over a certain timing, or a combination of voltages and timings therein. In some embodiments, the macromolecular block can modulate translocation of a residue across a readhead, thereby producing a distinguishable signal or signal break. In some embodiments, the macromolecular block can modulate enzymatic synthesis through steric hinderance to tune incorporation kinetics or limit the processivity of the enzyme. As a consequence, the macromolecular block can serve to attenuate translocation, attenuate incorporation kinetics or enzymatic processivity, or modulate and signal generated when an associated nucleotide passes through a readhead. In some embodiments, the macromolecular blocks can comprise various molecular entities, including but not limited to: oligonucleotide blocks, polyethylene glycol (PEG) blocks, peptide blocks, or chemical structures with incorporated fluorene elements.

In another aspect, the disclosed technology provides systems, devices, kits, and methods which allow for the attachment of a macromolecular block to a nucleotide, nucleotide backbone, translocating polymer, or other chemical group associated or attached to a sequence of nucleotides. Systems may be prepared to allow parallel reads in multiple nanopores, such as thousands or millions of nanopores. Accordingly, components of any system may be functionally duplicated to multiply sequencing throughput. Any system may also be adapted with microfluidics or automation. Any system may add one or multiple combinations of macromolecular blocks to any of nucleotides, nucleotide backbones, or structures correlating to nucleotide sequences.

The systems, devices, kits, and methods disclosed herein each have several aspects, no single one of which is solely responsible for their desirable attributes. Without limiting the scope of the claims, some prominent features are discussed herein. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

Additional details of exemplary nanopore sequencing devices which can be used with the disclosed technology, and methods of operating the devices, can be found in U.S. Provisional Patent Application Nos. 63/200,868 and 63/169,041, the entirety of each of the disclosures is incorporated herein by reference.

Disclosed herein is a modified nucleotide comprising a modification covalently attached to its nucleobase or its sugar, wherein the modification comprises a macromolecule block selected from the group consisting of an oligonucleotide block, a polyethylene glycol block, a peptide block, and a fluorinated block. In some embodiments, the macromolecular block is configured to slow, pause, or halt translocation. In some embodiments, the macromolecular blocks are associated with the target polynucleotide via hydrogen bonding, Van-der-Waals interactions, ionic interactions, or hydrophobic interactions.

In some embodiments, the oligonucleotide block comprises natural DNA/RNA nucleotides, modified nucleic acids, or a combination thereof. In some embodiments, the oligonucleotide block comprises a linear structure, a branched structure, a hairpin type structure, or a cyclic structure.

In some embodiments, the oligonucleotide block comprises natural or modified phosphate backbones which include phosphorothioates, phosphorodithioates, methylphosphonates, phosphoramidates, aminoalkylated phosphoramidates or a combination thereof. In some embodiments, the oligonucleotide block comprises interspersed spacer modifiers present in between bases. Example interspersed spacer modifiers are:

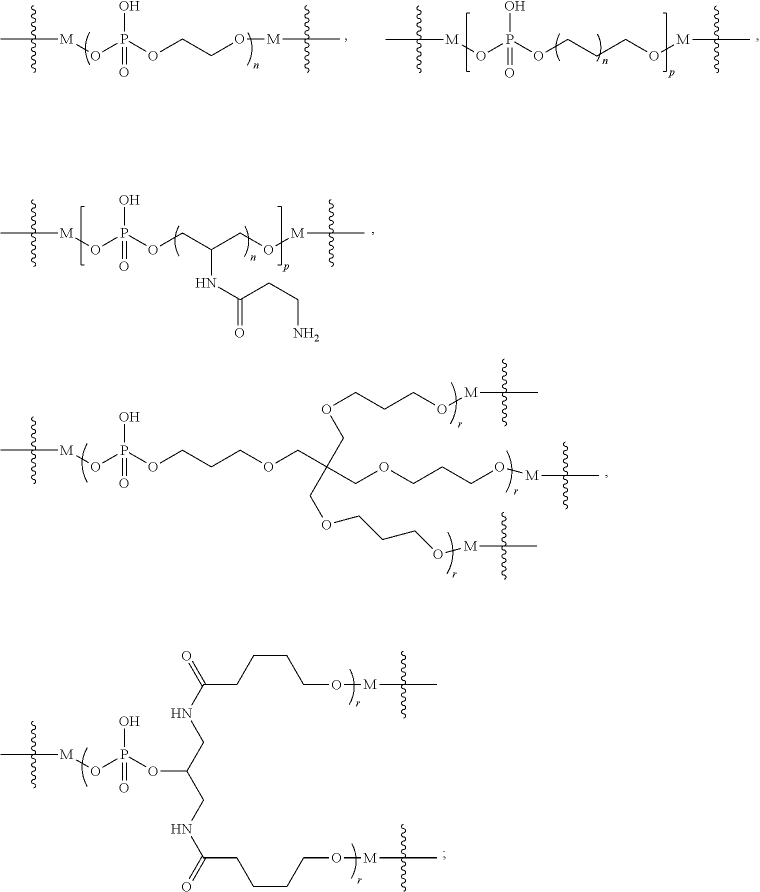

wherein each M is a natural DNA/RNA nucleotides, modified nucleic acids, or a combination thereof; p is an integer from 1-50; n is an integer from 1-30; and r is an integer 1-5, 6-10. However, spacer modifiers may be any spacer, whether hydrophilic or hydrophobic, and have any charge, whether positive, negative, or neutral.

In some embodiments, the oligonucleotide block includes a compound having one of the following structures, where a branching monomer is/are present:

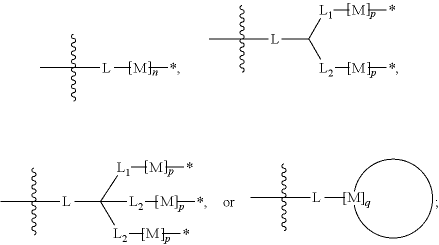

wherein each M is a natural nucleotide or a modified nucleic acid; n is an integer selected from 1 to 70 and, in some embodiments, n may be selected from 1-10, 11-30, 31-50, 51-70 or any interval between 1 and 70; p is an integer selected from 1 to 30 and, in some embodiments, p may be selected from 1-10, 11-20, 21-30 or any interval between 1 and 30; q is an integer selected from 2 to 10; and * is an end group. Examples of the end group * are

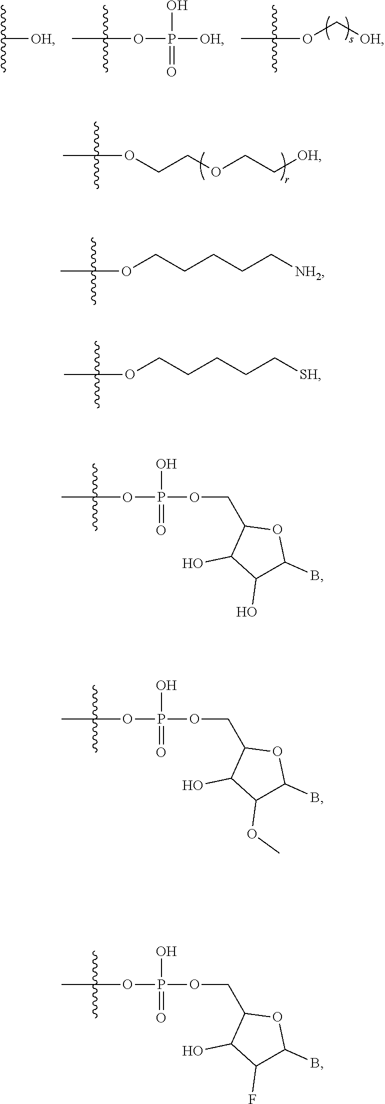

wherein r is 2, 3, 4, or 5, s is 3, 4, 5, 6, 7, 8, 9, 10, 11, or 12, and wherein “B” is Uracil, Cytosine, Guanine, or Adenine.

In some embodiments, the polyethylene block comprises a linear or a branched structure. In some embodiments, the polyethylene glycol block includes a compound having one of the following structures:

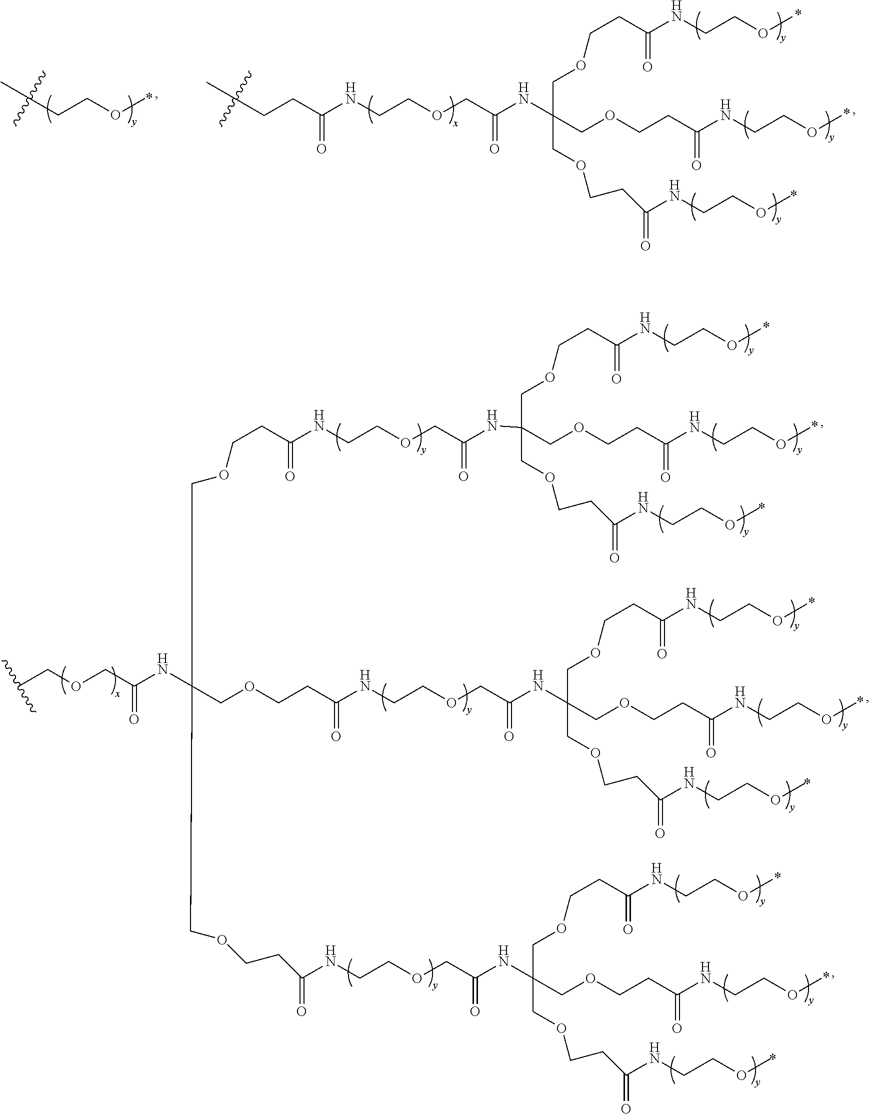

wherein x is an integer selected from 1 to 10; y is an integer selected from 1 to 30; and * is an end group selected from

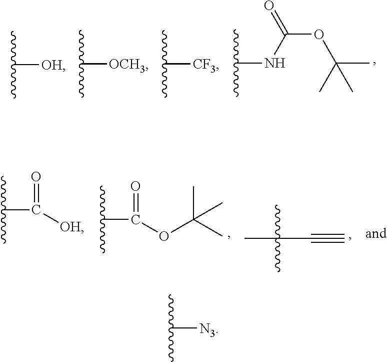

In some embodiments, the macromolecule block is a peptide block and the peptide block includes a compound having one of the following structures:

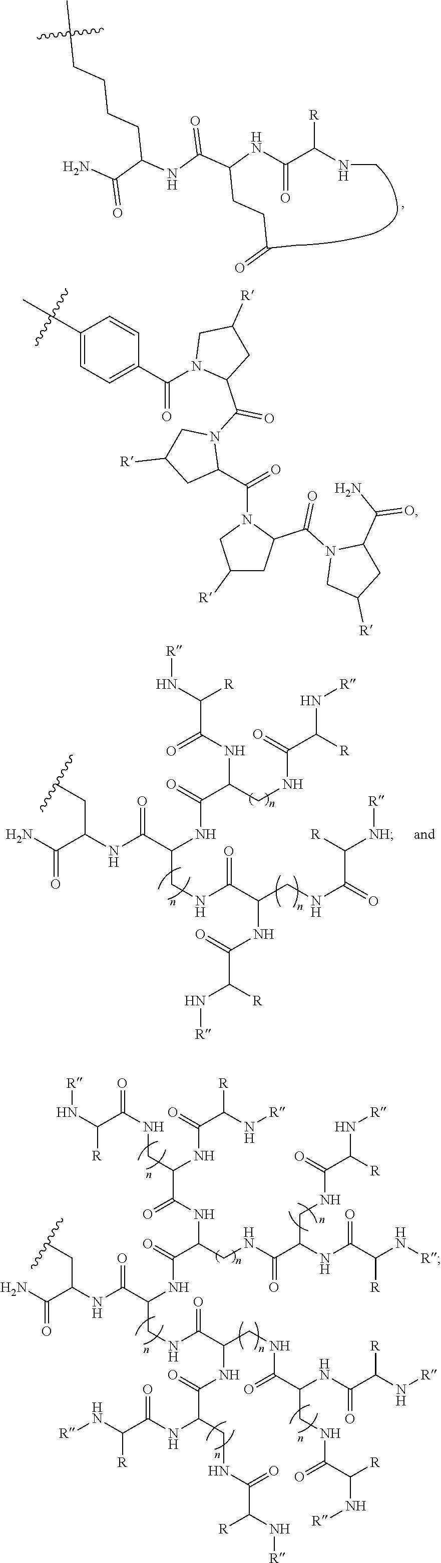

wherein each R is independently a side chain residue of an amino acid selected from:

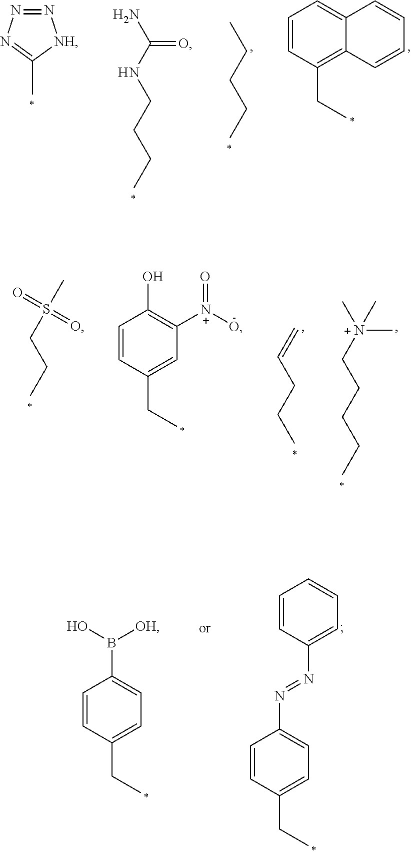

R′ is —OH, —CO₂, —OSO₃⁻, or —OPO₃²⁻; R″ is H, acyl, acryl, alloc, benzoyl, BOC, Fmoc, formyl, or Cbz; z is an integer from 3-6; and n is an integer from 1-5.

In some embodiments, the macromolecule block is a fluorinated block and the fluorinated block comprises a perfluoroalkyl block, fluoro-peptide block, or perfluoroalkylated oligo block. In some embodiments, the fluorinated block includes a compound having one of the following structures:

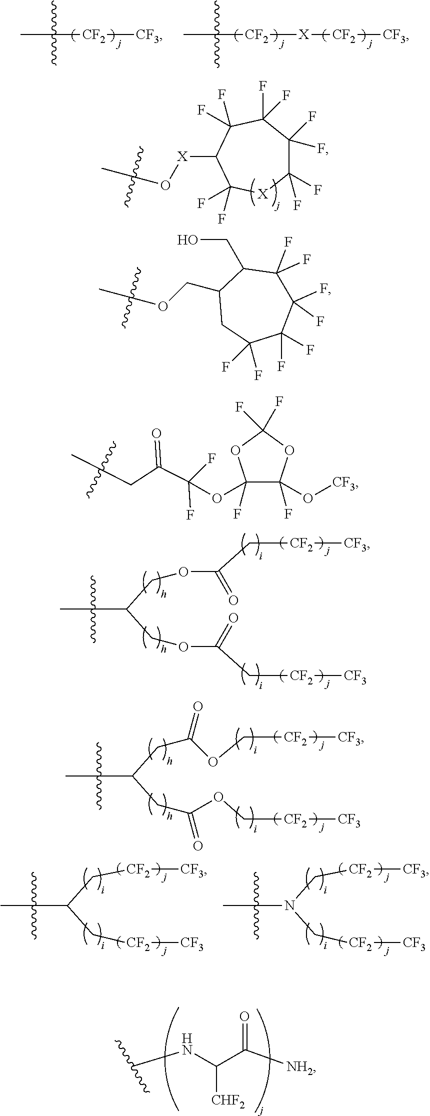

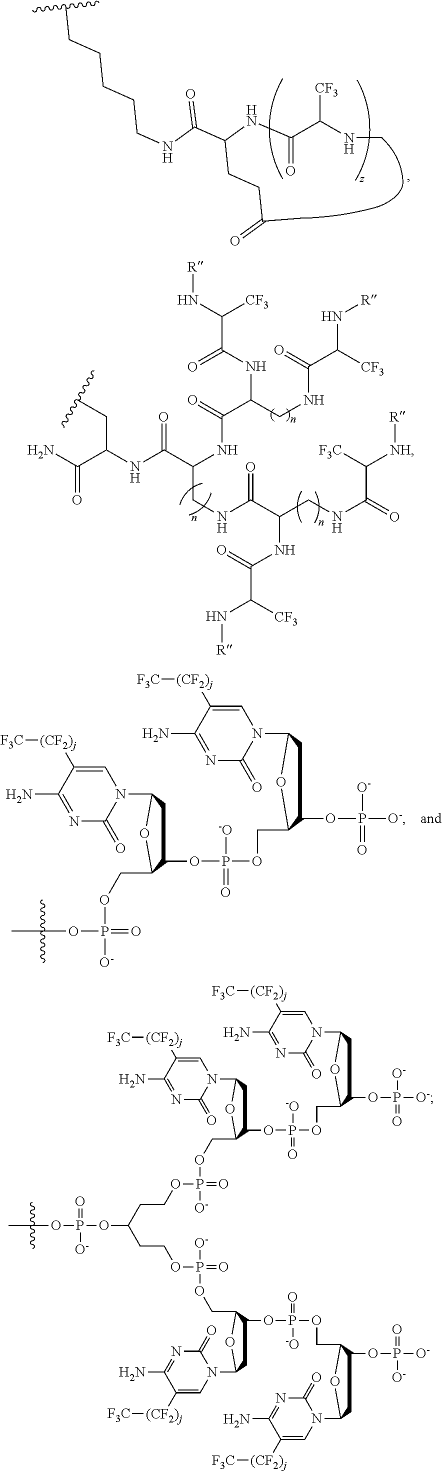

wherein h is an integer selected from 1-5, i is an integer selected from 1-5, j is an integer selected from 1-6; n is an integer selected from 1-5, z is an integer selected from 1-6; R″ is H, acyl, acryl, alloc, benzoyl, BOC, Fmoc, formyl, or Cbz, and X is C, O, PO₂, PO₃, or NR.

In some embodiments, the modified nucleotide further comprises a covalent coupling between the macromolecule block and the modified nucleotide, wherein the covalent coupling comprises a moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene. In some embodiments, the modification further comprises one or more covalently linked moieties selected from the group consisting of: dyes, synthetic polymers, and small molecules. In some embodiments, an oligonucleotide comprising one or more of the modified nucleotides is provided.

In some embodiments, a method for determining a sequence of a target polynucleotide in a nanopore-based sequencing system is also provided, the method comprising: providing a daughter strand of a target polynucleotide comprising one or more modified nucleotides, reading the modified nucleotides by applying a reading voltage across a read head to identify a first reporter element in a constriction of a nanopore based on a first electrical response in the system, wherein one or more nucleotides translocate through a nanopore.

In some embodiments, an additional method for determining a sequence of a target polynucleotide in a nanopore-based sequencing system is provided. The method comprises providing the target polynucleotide and a plurality of the macromolecule blocks in an electrolyte for the nanopore-based sequencing system; applying a voltage bias to cause the target polynucleotide to translocate through a constriction of a nanopore, wherein one or more macromolecule blocks are non-covalently associated with the target polynucleotide; and detecting and identifying one or more nucleotides as the nucleotides pass through the constriction based on an electrical response in the system.

Disclosed herein further includes a kit for performing a method for determining a sequence of a polynucleotide in a nanopore-based sequencing system, the kit comprising the compound disclosed herein.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIGS. 1A and 1B schematically illustrate an example of interactions of macromolecular blocks with residues of a nanopore or with the translocating polymer.

FIG. 2 schematically illustrates examples of PEG blocks.

FIG. 3 schematically illustrates examples of oligonucleotide blocks.

FIG. 4 schematically illustrates an example of peptide blocks.

FIG. 5 schematically illustrates an example of fluorinated blocks.

FIGS. 6A and 6B illustrate translocation events with various peptide blocks.

FIG. 7 illustrates translocation events with various PEG blocks.

FIG. 8 illustrates translocation events with various Oligonucleotide blocks/ratchets.

FIG. 9 illustrates modifications to the oligonucleotide and waveform when bound to peptide block B4E.

FIG. 10 illustrates modifications to ratcheting efficiency as a function of pulse duration.

FIGS. 11A, 11B, 12A, and 12B illustrate molecular dynamic simulations of proposed peptide macromolecular blocks.

FIG. 13 illustrates molecular dynamic simulations of proposed macromolecular blocks.

FIG. 14 illustrates a plot of ratcheting efficiency as a function of pulse width and magnitude for B4E and B4Gla.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores. In some embodiments, a refers to a pore having an opening with a diameter at its most narrow point of about 0.3 nm to about 2 nm. For example, a nanopore may be a solid-state nanopore, a graphene nanopore, an elastomer nanopore, or may be a naturally-occurring or recombinant protein that forms a tunnel upon insertion into a bilayer, thin film, membrane, or solid-state aperture, also referred to as a protein pore or protein nanopore herein (e.g., a transmembrane pore). If the protein inserts into the membrane, then the protein is a tunnel-forming protein.

As used herein, the term “diameter” is intended to mean a longest straight line inscribable in a cross-section of a nanoscale opening through a centroid of the cross-section of the nanoscale opening. It is to be understood that the nanoscale opening may or may not have a circular or substantially circular cross-section (the cross-section of the nanoscale opening being substantially parallel with the cis/trans electrodes). Further, the cross-section may be regularly or irregularly shaped.

As used herein, “cis” refers to the side of a nanopore opening through which an analyte or modified analyte enters the opening or across the face of which the analyte or modified analyte moves.

As used herein, “trans” refers to the side of a nanopore opening through which an analyte or modified analyte (or fragments thereof) exits the opening or across the face of which the analyte or modified analyte does not move.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, a “moiety” is one of two or more parts into which something may be divided, such as, for example, the various parts of a tether, a molecule or a probe.

As used herein, a “reporter” is composed of one or more reporter elements. Reporters include what are known as “tags” and “labels.” The linker construct (when including reporter moiety) or nucleobase residue of the elongated polymer can be considered a reporter. Reporters serve to parse the genetic information of the target nucleic acid.

As used herein, a “linker” is a molecule or moiety that joins two molecules or moieties and provides spacing between the two molecules or moieties such that they are able to function in their intended manner. For example, a linker can comprise a diamine hydrocarbon chain that is covalently bound through a reactive group on one end to an oligonucleotide analog molecule and through a reactive group on another end to a solid support, such as, for example, a bead surface. Coupling of linkers to nucleotides and substrate constructs of interest can be accomplished through the use of coupling reagents that are known in the art (see, e.g., Efimov et al., Nucleic Acids Res. 27:4416-4426, 1999). Methods of derivatizing and coupling organic molecules are well known in the arts of organic and bioorganic chemistry. A linker may also be cleavable or reversible.

As used herein, the term “polypeptide nanopore” is intended to mean a protein/polypeptide that extends across the membrane, and permits ions, electric current, polymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an a-helix bundle nanopore and a B-barrel nanopore. Example polypeptide nanopores include a-hemolysin, Mycobacterium smegmatis porin A (MspA), gramicidin A, maltoporin, OmpF, OmpC, PhoE, Tsx, F-pilus, etc. The protein a-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

As used herein, a “peptide” refers to two or more amino acids joined together by an amide bond (that is, a “peptide bond”). Peptides may be linear or cyclic. Peptides may be α, β, γ, δ, or higher, or mixed. Peptides may comprise any mixture of amino acids as defined herein, such as comprising any combination of D, L, α, β, γ, δ, or higher amino acids.

As used herein, a “protein” refers to an amino acid sequence having multiple linked amino acids.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

The nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

The application of the electric potential difference across a nanopore may force the translocation of a nucleic acid through the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the constriction, for example. The signal from that change in current can be measured using any of a variety of methods. Each signal is unique to the species of nucleotide(s) (or linker constructs with a reporter moiety region) in the nanopore, such that the resultant signal can be used to determine a characteristic of the polynucleotide. For example, the identity of one or more species of nucleotide(s) (or probe) that produces a characteristic signal can be determined.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothiolate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, voltage, conductance, or a transverse electrical effect. An “electronic current” or “electric current” refers to a flow of electric charge. In an example, an electrical signal may be an electric current passing through a nanopore, and the electric current may flow when an electric potential difference is applied across the nanopore.

As used herein, the term “driving force” is intended to mean an electrical current that allows a polynucleotide to translocate through the nanopore. In some embodiments, the electrical current electric current may flow when an electric potential difference is applied across the nanopore.

As used herein, the term “holding force” is intended to mean a resistance that slows and/or stops a polynucleotide to translocate through the nanopore. In some embodiments, the holding force is overcome by the application of a driving force. Thus, the driving force overcomes/overrides the resistance that slows and/or stops a polynucleotide, thereby allowing the polynucleotide to translocate through the nanopore.

As used herein, the terms “modification,” “block,” or “macromolecular block” are intended to refer to any group or moiety attached or interacting with a nucleotide or polymer backbone. A macromolecular block provides resistance that can attenuate both the signal, and the rate of signal generation, when a polymer transduces across a readhead. The resistance provided by the block is due to inherent chemical and physical properties, (e.g., size, geometry, and/or non-covalent interactions with residues comprising the nanopore). Blocks can operate as a ratchet or a brake for the polypeptide translocation through a nanopore. A block can be attached to any part of the nucleotide and can also be attached to the nucleotide at two locations forming a loop.

As used herein, the term “end group” should be interpreted as would be understood by a person having ordinary skill in the art but can also refer to a terminal chemical group on a PEG ratchet. An end group can interact with residues of the pore or with each other to affect translocation of DNA.

As used herein, “alloc” is intended to mean Allyloxycarbonyl, “BOC” is intended to mean butyloxycarbonyl, “Fmoc” is intended to mean fluorenylmethoxycarbonyl, and “Cbz” is intended to mean benzyloxycarbonyl. In some embodiments these can function as protecting groups.

The aspects and examples set forth herein and recited in the claims can be understood in view of the above definitions.

### Overview

Nanopore sequencing of nucleic acids typically involves the movement of a polynucleotide, or a polymeric chain corresponding to a polynucleotide, through an embedded nanopore. The movement of the nucleotides or associated moieties therefore generates an attenuated electrical signal, which can be processed in silico to instantiate any underlying nucleotide sequence. However, nanopore sequencers are sensitive to multiple bases of a polynucleotide strand resident in the nanopore. As opposed to reading a single base one at a time, multiple nucleotides can generate multiple signals, which are indistinct and require complex signal deconvolution. For example, the MspA nanopore possesses a constriction region which serves as a readhead of at least 4 nucleotides (a “k-mer”), resulting in minimally 256 (4\hat{ }4) different permutation of 4-mer sequences that need to be deconvoluted. For a k-mer of 5 bases, the number of possible signals increases to 4\hat{ }5=1,024. A longer readhead will result in an exponential increase in the number of signals to be differentiated, which complicates the sequencing readout and increases the complexity of base calling, thus reducing accuracy. Another issue with nanopore sequencers is that the speed of translocation of natural single stranded DNA is on the order of >10 million nucleotides per second, far above the rate that is compatible with electronics and detectors.

In some embodiments, disclosed herein is a modified nucleotide comprising a modification covalently attached to its nucleobase or its sugar, wherein the modification comprises a macromolecule block selected from the group consisting of an oligonucleotide block, a polyethylene glycol block, a peptide block, and a fluorinated block. In some embodiments, the macromolecular block is configured to slow, pause, or halt translocation. In some embodiments, the macromolecular blocks are associated with the target polynucleotide via hydrogen bonding, Van-der-Waals interactions, ionic interactions, or hydrophobic interactions.

Wherein the oligonucleotide block includes a compound having one of the following structures:

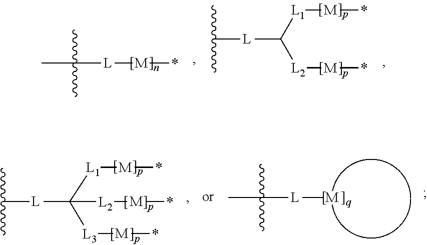

wherein each M is a natural nucleotide or a modified nucleic acid;
n is an integer selected from 1 to 70 and, in some embodiments, n may be selected from 1-10, 11-30, 31-50, 51-70 or any interval between 1 and 70;
p is an integer selected from 1 to 30 and, in some embodiments, p may be selected from 1-10, 11-20, 21-30 or any interval between 1 and 30;
q is an integer selected from 2 to 10; and
* is an end group selected from the group consisting of

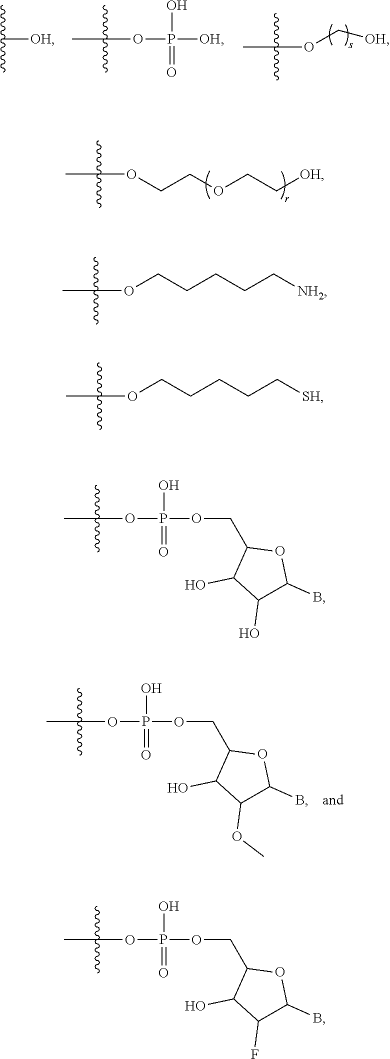

wherein r is 2, 3, 4, or 5; s is 3, 4, 5, 6, 7, 8, 9, 10, 11, or 12; and “B” is Uracil, Cytosine, Guanine, or Adenine.

Wherein the polyethylene glycol block includes a compound having one of the following structures:

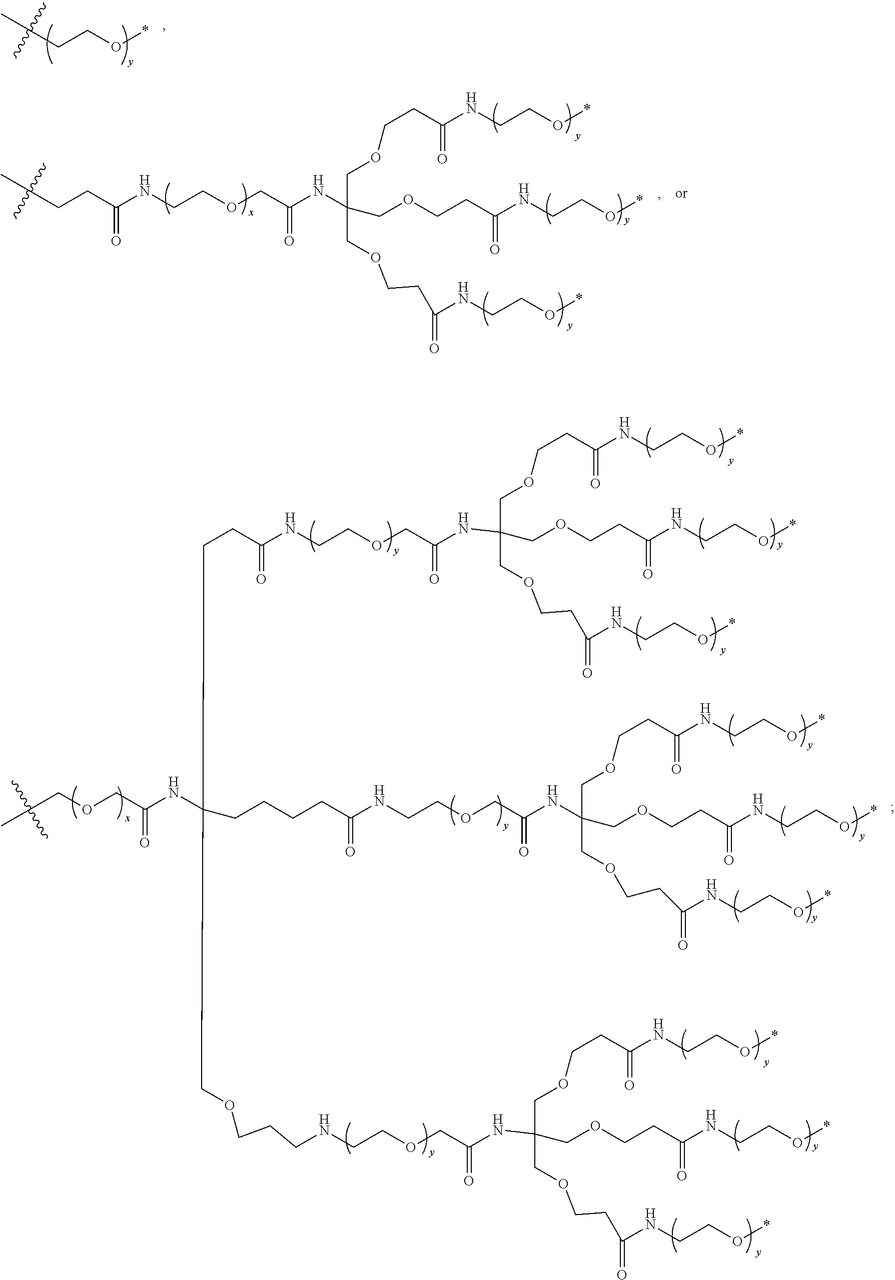

wherein x is an integer selected from 1 to 10;
y is an integer selected from 1 to 30; and
* is an end group selected from the group consisting of

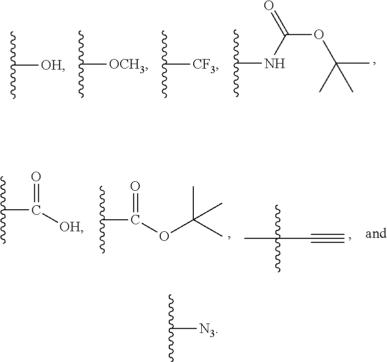

Wherein the peptide block includes a compound having one of the following structures:

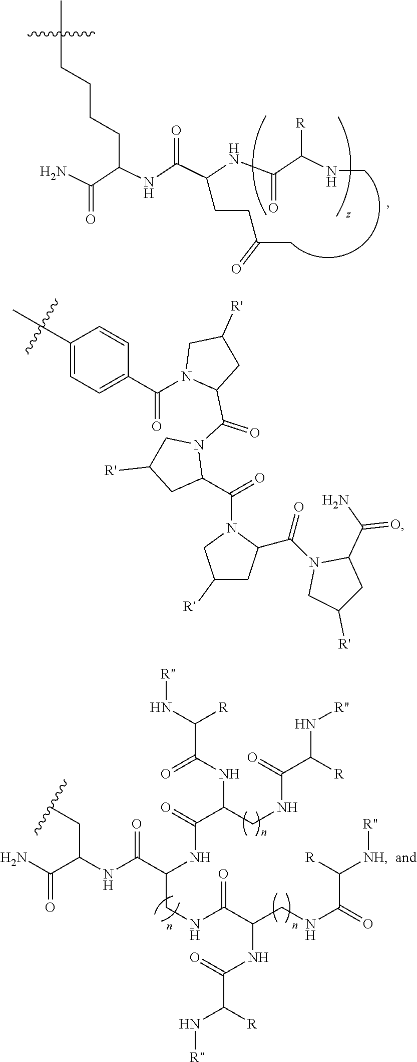

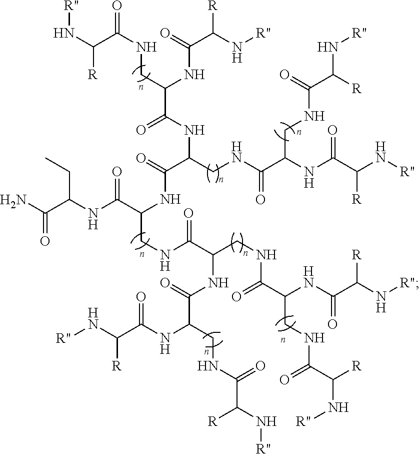

wherein each R is independently a side chain residue of an amino acid selected from the group consisting of

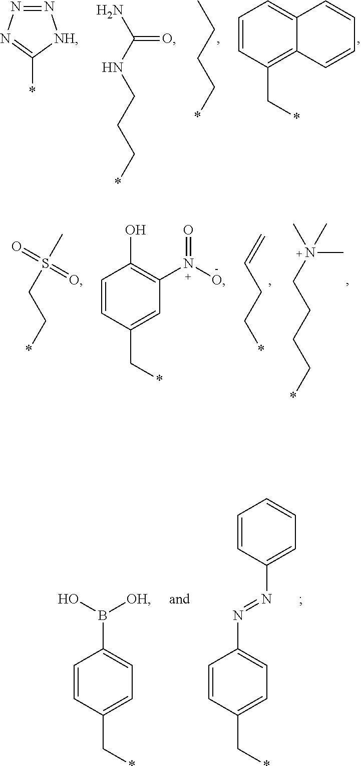

R′ is —OH, —CO₂⁻, —OSO₃⁻, or —OPO₃²⁻; R″ is H, acyl, acryl, alloc, benzoyl, BOC, Fmoc, formyl, or Chz, and z is an integer from 3-6; and n is an integer from 1-5.

Wherein the fluorinated block includes a compound having one of the following structures:

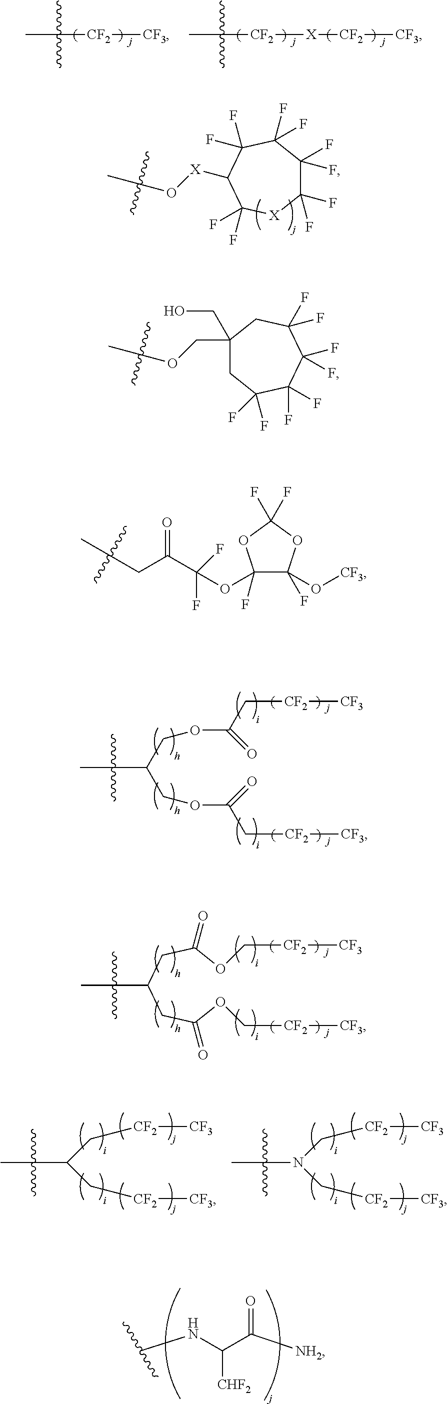

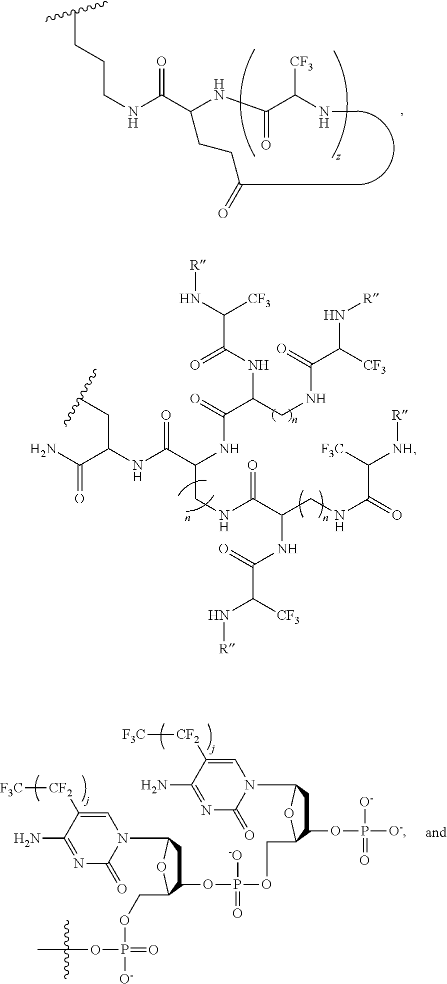

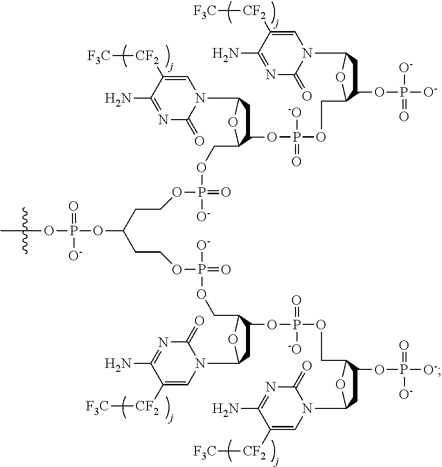

wherein h is an integer selected from 1 to 5, i is an integer selected from 1 to 5, j is an integer selected from 1 to 6; n is an integer selected from 1 to 5, z is an integer selected from 1 to 6; R″ is H, acyl, acryl, alloc, benzoyl, BOC, Fmoc, formyl, or Cbz; and X is C, O, PO₂, PO₃, or NR. In some embodiments the h, i, j, and z values are determined by balancing the composition of fluorocarbon to hydrocarbons within blocks.

In some embodiments, the oligonucleotide block comprises natural DNA/RNA nucleotides, modified nucleic acids, or a combination thereof. In some embodiments, the oligonucleotide block comprises a linear structure, a branc63,106hed structure, a hairpin type structure, or a cyclic structure. In some embodiments, the polyethylene block comprises a linear or a branched structure. In some embodiments, the fluorinated block comprises a perfluoroalkyl block, fluoro-peptide block, or perfluoroalkylated oligo block.

In some embodiments, the modified nucleotide further comprises a covalent coupling between the macromolecule block and the modified nucleotide, wherein the covalent coupling comprises a moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene. In some embodiments, the modification further comprise one or more covalently linked moieties selected from the group consisting of dyes, synthetic polymers, and small molecules. In some embodiments, an oligonucleotide comprising one or more of the modified nucleotides is provided.

In some embodiments, the oligonucleotide block comprises natural or modified phosphate backbones which include phosphorothioates, phosphorodithioates, methylphosphonates, phosphoramidates, aminoalkylated phosphoramidates or a combination thereof. In some embodiments, the oligonucleotide block comprises interspersed spacer modifiers present in between bases. Examples of interspersed spacer modifiers are:

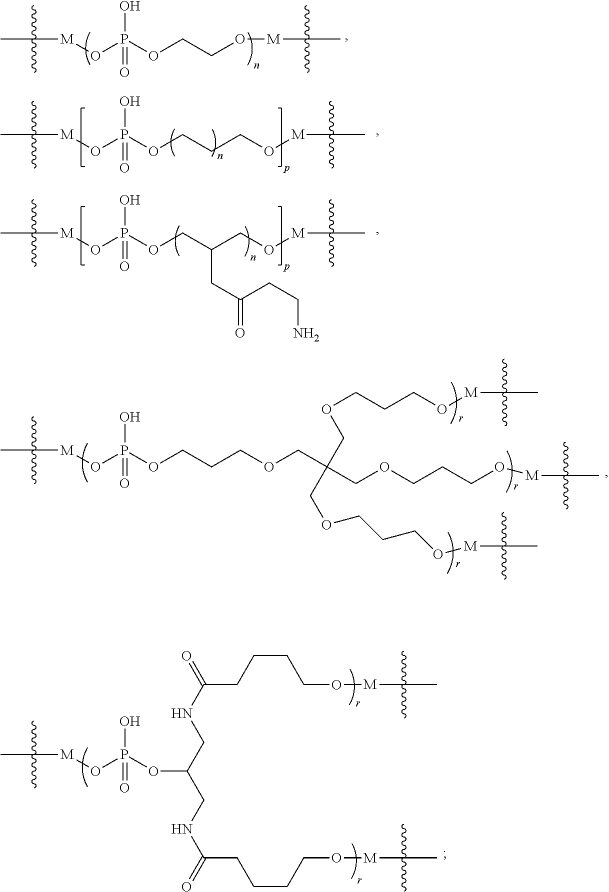

wherein each M is a natural DNA/RNA nucleotide, modified nucleic acid, or a combination thereof; p is an integer from 1-50; n is an integer from 1-30; and r is an integer from 1-5, or 6-10. However, spacer modifiers may be any spacer, whether hydrophilic or hydrophobic, and have any charge, whether positive, negative, or neutral.

In some embodiments, a method for determining a sequence of a target polynucleotide in a nanopore-based sequencing system is also provided, the method comprising: providing a daughter strand of a target polynucleotide comprising one or more modified nucleotides, reading the modified nucleotides by applying a reading voltage across a read head to identify a first reporter element in a constriction of a nanopore based on a first electrical response in the system, wherein one or more nucleotides translocate through a nanopore.

In some embodiments, an additional method for determining a sequence of a target polynucleotide in a nanopore-based sequencing system is provided. The method comprises providing the target polynucleotide and a plurality of the macromolecule blocks in an electrolyte for the nanopore-based sequencing system; applying a voltage bias to cause the target polynucleotide to translocate through a constriction of a nanopore, wherein one or more macromolecule blocks are non-covalently associated with the target polynucleotide; and detecting and identifying one or more nucleotides as the nucleotides pass through the constriction based on an electrical response in the system.

Disclosed herein further includes a kit for performing a method for determining a sequence of a polynucleotide in a nanopore-based sequencing system, the kit comprising the compound disclosed herein.

### System and Method

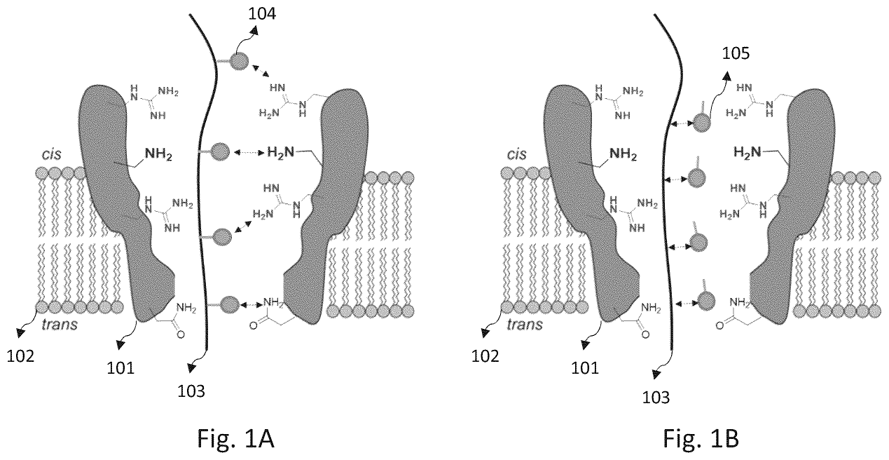

FIGS. 1A and 1B schematically illustrate examples of the interaction of macromolecular blocks with either residues of the MspA nanopore (FIG. 1A) or with the translocating polymer (FIG. 1B). A nanopore 101 is deposited in a lipid bilayer 102. An elongated polymer/polynucleotide 103 translocates through the nanopore 101. The elongated polymer/polynucleotide 103 includes bound macromolecular blocks 104 (FIG. 1A), or associated macromolecular blocks 105 (FIG. 1B). By introducing a macromolecular block, the signal can be attenuated due to interactions of the block with residues comprising the nanopore, resulting in fewer and more isolated signals (for A, T, C and G), reducing the complexity of base calling. A characteristic linker/barcode may be assigned to each of the macromolecular blocks or bases to achieve base recognition.

By “translocation,” it is meant that an analyte (e.g., DNA) enters one side of an opening of a nanopore and moves to and out of the other side of the opening. It is contemplated that any embodiment herein comprising translocation may refer to electrophoretic translocation or non-electrophoretic translocation, unless specifically noted. An electric field may move an analyte (e.g., a polynucleotide) or modified analyte. By “interacts,” it is meant that the analyte (e.g., DNA) or modified analyte moves into and, optionally, through the opening, where “through the opening” (or “translocates”) means to enter one side of the opening and move to and out of the other side of the opening. Optionally, methods that do not employ electrophoretic translocation are contemplated. In some embodiments, physical pressure causes a modified analyte to interact with, enter, or translocate (after alteration) through the opening. In some embodiments, a magnetic bead is attached to an analyte or modified analyte on the trans side, and magnetic force causes the modified analyte to interact with, enter, or translocate (after alteration) through the opening. Other methods for translocation include, but are not limited, to gravity, osmotic forces, temperature, and other physical forces such as centripetal force.

In some embodiments, the nanopore may comprise a solid-state material, such as silicon nitride, modified silicon nitride, silicon, silicon oxide, or graphene, or a combination thereof. In some embodiments, the nanopore is a protein that forms a tunnel upon insertion into a bilayer, membrane, thin film, or solid-state aperture. In some embodiments, the nanopore is disposed in a lipid bilayer. In some embodiments, the nanopore is disposed in an artificial membrane comprising a mycolic acid. The nanopore may be a Mycobacterium smegmatis porin (Msp) having a vestibule and a constriction zone that define the tunnel. The Msp porin may be a mutant MspA porin. In some embodiments, amino acids at positions 90, 91, and 93 of the mutant MspA porin are each substituted with asparagine. Some embodiments may comprise altering the translocation velocity or sequencing sensitivity by removing, adding, or replacing at least one amino acid of an Msp porin. A “mutant MspA porin” is a multimer complex that has at least or at most 70, 75, 80, 85, 90, 95, 98, or 99 percent or more identity, or any range derivable therein, but less than 100%, to its corresponding wild-type MspA porin and retains tunnel-forming capability. A mutant MspA porin may be a recombinant protein. Optionally, a mutant MspA porin is one having a mutation in the constriction zone or the vestibule of a wild-type MspA porin. Optionally, a mutation may occur in the rim or the outside of the periplasmic loops of a wild-type MspA porin. A mutant MspA porin may be employed in any embodiment described herein.

A “vestibule” refers to the cone-shaped portion of the interior of an Msp porin whose diameter generally decreases from one end to the other along a central axis, where the narrowest portion of the vestibule is connected to the constriction zone. A vestibule may also be referred to as a “goblet.” The vestibule and the constriction zone together define the tunnel of an Msp porin. A “constriction zone” or the “readhead” refers to the narrowest portion of the tunnel of an Msp porin, in terms of diameter, that is connected to the vestibule. The length of the constriction zone may range from about 0.3 nm to about 2 nm. Optionally, the length is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. The diameter of the constriction zone may range from about 0.3 nm to about 2 nm. Optionally, the diameter is about, at most about, or at least about 0.3, 0.4, 0.5, 0.6, 0.7, 0.8, 0.9, 1.0, 1.1, 1.2, 1.3, 1.4, 1.5, 1.6, 1.7, 1.8, 1.9, 2, or 3 nm, or any range derivable therein. A “tunnel” refers to the central, empty portion of an Msp porin that is defined by the vestibule and the constriction zone, through which a gas, liquid, ion, or analyte may pass. A tunnel is an example of an opening of a nanopore.

Various conditions such as light and the liquid medium that contacts a nanopore, including its pH, buffer composition, detergent composition, and temperature, may affect the behavior of the nanopore, particularly with respect to its conductance through the tunnel as well as the movement of an analyte with respect to the tunnel, either temporarily or permanently.

In some embodiments, the disclosed system for nanopore sequencing comprises an Msp porin having a vestibule and a constriction zone that define a tunnel, wherein the tunnel is positioned between a first liquid medium and a second liquid medium, wherein at least one liquid medium comprises an analyte polynucleotide, and wherein the system is operative to detect a property of the analyte. The system may be operative to detect a property of any analyte comprising subjecting an Msp porin to an electric field such that the analyte interacts with the Msp porin. The system may be operative to detect a property of the analyte comprising subjecting the Msp porin to an electric field such that the analyte electrophoretically translocates through the tunnel of the Msp porin. In some embodiments, the system comprises an Msp porin having a vestibule and a constriction zone that define a tunnel, wherein the tunnel is positioned in a lipid bilayer between a first liquid medium and a second liquid medium, and wherein the only point of liquid communication between the first and second liquid media occurs in the tunnel. Moreover, any Msp porin described herein may be comprised in any system described herein. In some embodiments, the system may further comprise an amplifier or a data acquisition device. The system may further comprise one or more temperature regulating devices in communication with the first liquid medium, the second liquid medium, or both. The system described herein may be operative to translocate an analyte through an Msp porin tunnel either electrophoretically or otherwise.

In some embodiments, macromolecular blocks can also modulate enzymatic synthesis through steric hindrance to tune incorporation kinetics or limit the processivity of an incorporating enzyme, including polymerase. Various polymerases exist generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase I, Klenow fragment, Thermophilus aquaticus DNA polymerase, Tth DNA polymerase, VentR® DNA polymerase (New England Biolabs), Deep VentR® DNA polymerase (New England Biolabs), Bst DNA Polymerase Large Fragment, Stoeffel Fragment, 90N DNA Polymerase, 90N DNA polymerase, Pfu DNA Polymerase, TfI DNA Polymerase, Tth DNA Polymerase, RepliPHI Phi29 Polymerase, TIi DNA polymerase, eukaryotic DNA polymerase beta, telomerase, Therminator™ polymerase (New England Biolabs), KOD HiFi™ DNA polymerase (Novagen), KODI DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178, 6,602,695, and 6,395,524 (incorporated by reference). These polymerases include wild-type, mutant isoforms, and genetically engineered variants. “Encode” or “parse” are verbs referring to transferring from one format to another, and refers to transferring the genetic information of target template base sequence into an arrangement of reporters.

### Modified Nucleotide

Disclosed herein is a modified nucleotide comprising a modification covalently attached to its nucleobase or its sugar, wherein the modification comprises a macromolecule block selected from the group consisting of an oligonucleotide block, a polyethylene glycol block, a peptide block, and a fluorinated block. In some embodiments, the macromolecular block is configured to slow, pause, or halt translocation. In some embodiments, the macromolecular blocks are associated with the target polynucleotide via hydrogen bonding, Van-der-Waals interactions, ionic interactions, or hydrophobic interactions.

### Oligonucleotide Blocks

Oligonucleotide blocks (ONBs) are macromolecular blocks designed to contain one or a series of nucleic acids that can interact strongly to charged residues within a nanopore. For MspA, the positively charged arginine and lysine residues can electrostatically and sterically affect translocation speed. ONBs can affect signal generation by methods including but not limited to interactions with residues at or near the constriction of the nanopore, interactions with translocating polymer or a combination of both. These interactions can be via H-bonding, Van-der-Waals interaction, ionic interaction or hydrophobic interactions. These interactions affect the flow of ions and consequently produce differential blockage current signatures.

In some embodiments, the ONB comprises natural DNA/RNA nucleotides, modified nucleic acids, or a combination thereof. The oligonucleotide block may comprise a linear structure, a branched structure, a hairpin type structure, or a cyclic structure. In some embodiments, the oligonucleotide block includes a compound having one of the following structures:

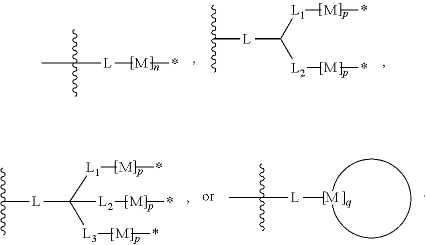

Each M is a natural nucleotide or a modified nucleic acid. n is an integer selected from 1 to 70, and in some embodiments, n may be selected from 1-10, 11-30, 31-50, 51-70, or any interval between 1 and 70. p is an integer selected from 1 to 30. In some embodiments, p may be selected from 1-10, 11-20, 21-30, or any interval between 1 and 30. q is an integer selected from 2 to 10; and * is an end group selected from the group consisting of

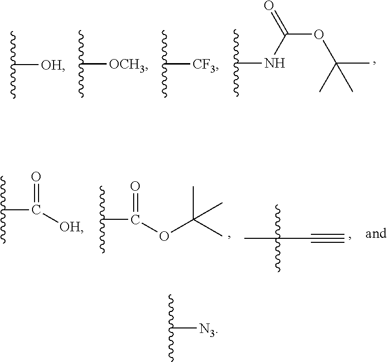

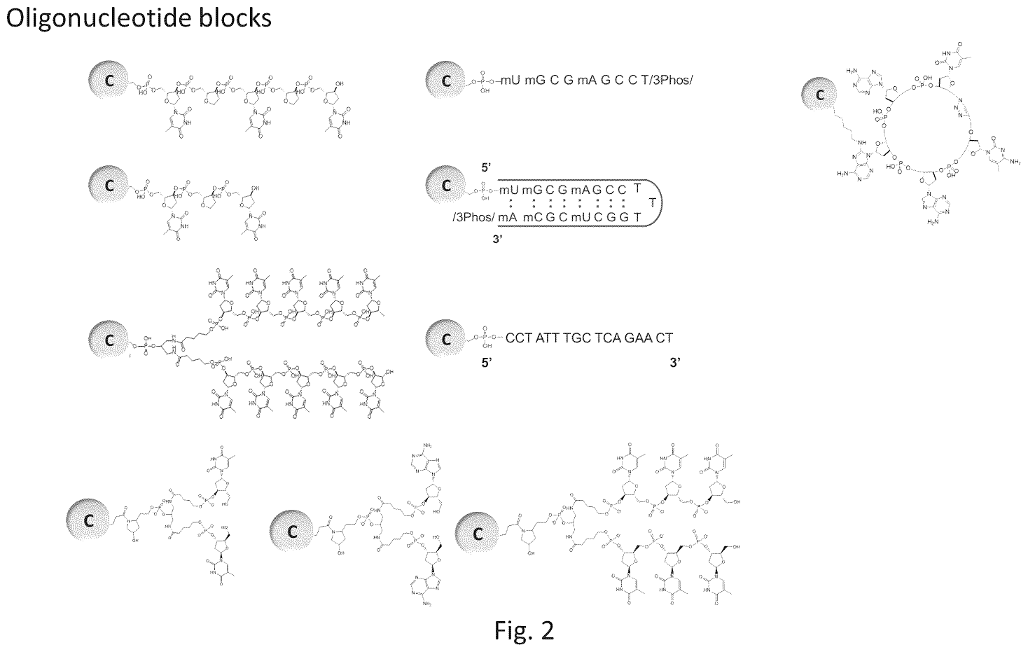

In some embodiments, non-limiting examples of ONBs are shown in FIG. 2.

FIG. 2 provides some non-limiting examples of linear, branched and hairpin type synthesized ONBs. Some ONBs contain modifications such as 2′-OMe nucleotide, abasic nucleotides and 3′ phosphate. ONBs can be linked to the translocating polymer or an enzyme substrate via C, where C refers to covalent coupling such as amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene. Some non-limiting examples of ONB's include:

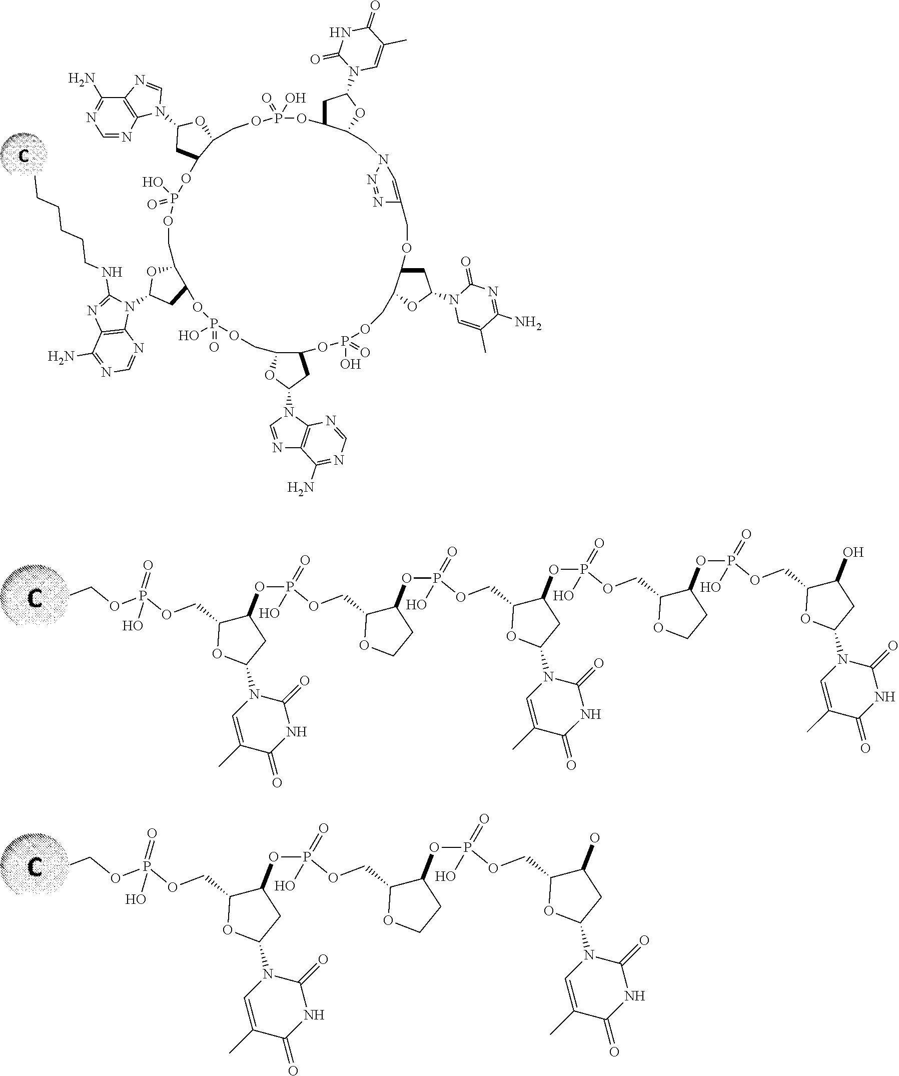

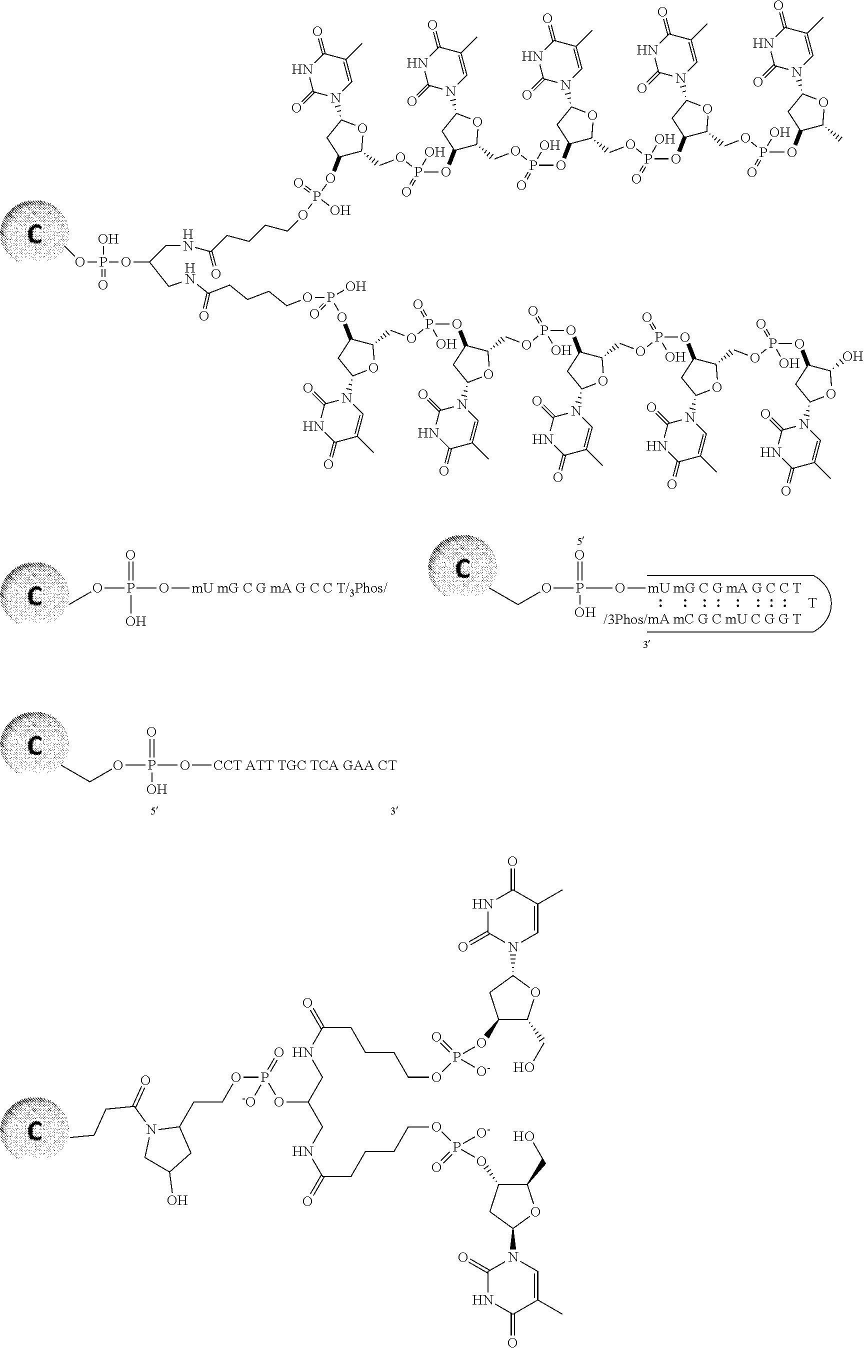

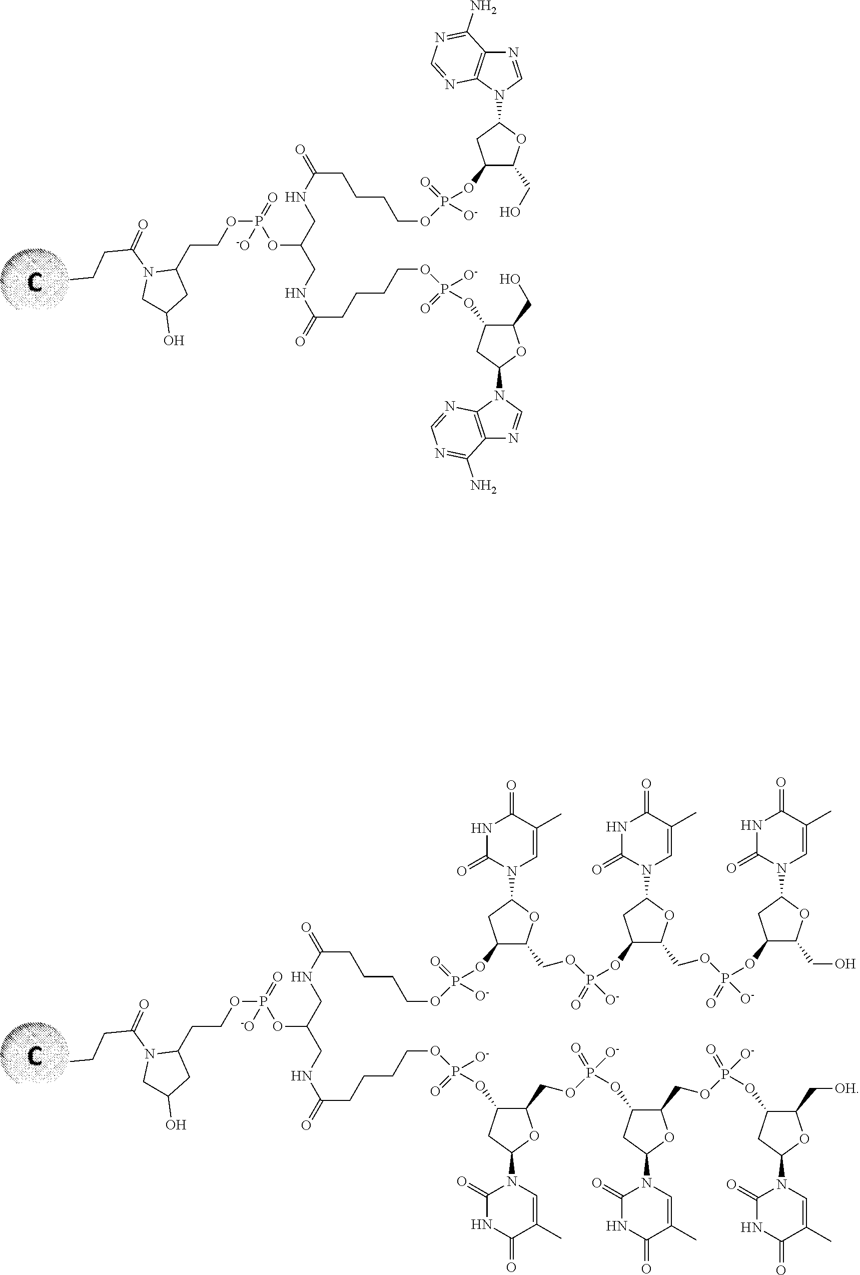

In some iterations, the ONB can be covalently attached to the translocating polymer and interacts with the pore residues (FIG. 1A). In some iterations, the ONB associates with the translocating polymer via non-covalent interactions (FIG. 1B). ONBs can be pulled into the nanopore vestibule either by associating with the translocating polymer or by the applied potential difference across the pore.

ONBs can also modulate enzymatic synthesis through steric hinderance to tune incorporation kinetics or limit the processivity of the enzyme. Nucleotide substrates for DNA/RNA polymerases can be modified with ONB at the nucleobase or at the ribose ring. In some iterations, the ONB remains on the nucleotide after incorporation. In some iterations, the ONB can be removed via specific biochemical bond cleavage. ONBs for modulating translocation, signal generation, and enzymatic synthesis can also be in single stranded form. They can either adopt linear, secondary or tertiary structures such as duplexes, triplexes, hairpins, G-quadruplexes, i-motifs, among others. ONBs can contain natural DNA/RNA nucleotides, modified nucleic acids or a combination of both. ONBs can also contain covalently linked modifications including but not limited to dyes, synthetic polymers, barcodes, or organic small molecules, among others.

### Polyethylene Glycol (PEG) Blocks

PEG Blocks are macromolecular blocks designed to one or a series of organic synthetic polymers with diverse chemical functional groups capable of non-covalent interactions with the nanopore. For MspA, the positively charged arginine and lysine residues can electrostatically and sterically affect translocation speed. Additionally, PEG constituent moieties can affect translocation speed, including for example, amide and cationic nitrogen moieties, as well as aromatic and aliphatic carbon moieties. PEG blocks can affect signal generation by methods including but not limited to interactions with residues at or near the constriction of the nanopore, interactions with translocating polymer or a combination of both. Like other blocks described herein, PEG block interactions can be via H-bonding, Van-der-Waals interaction, ionic interaction or hydrophobic interactions. Moreover, interactions can also arise due to the size or geometry of the block. These interactions affect the flow of ions and consequently produce differential blockage current signatures. Blocks herein, including PEG blocks, can also change the conformation or size of the association or attached polymer segment. In some embodiments, blocks described herein, including PEG blocks, can translocate through the pore.

In some embodiments, PEG blocks as described herein include a compound having one of the following structures:

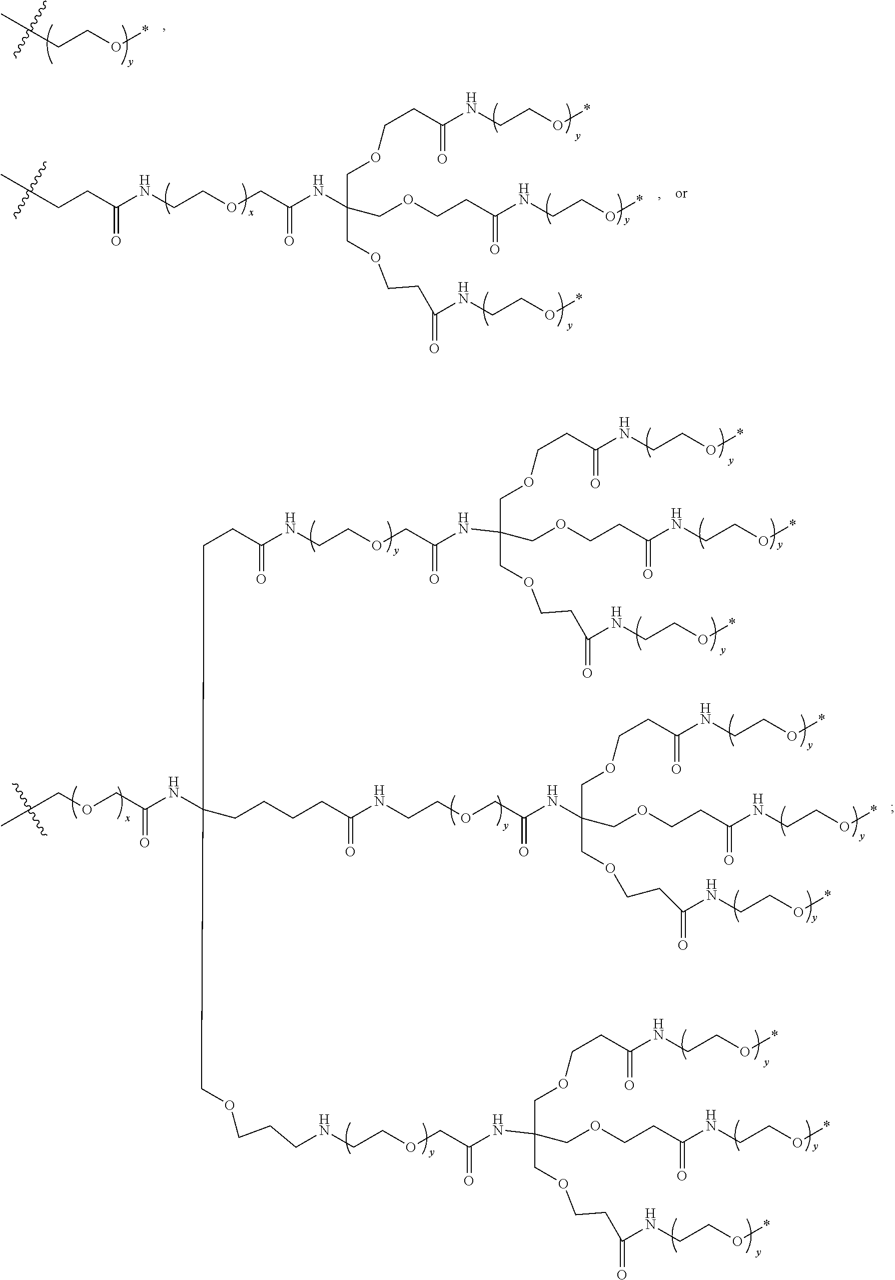

wherein x is an integer selected from 1 to 10; y is an integer selected from 1 to 30; and * is an end group selected from the group consisting of

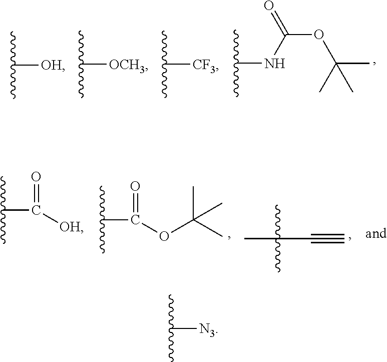

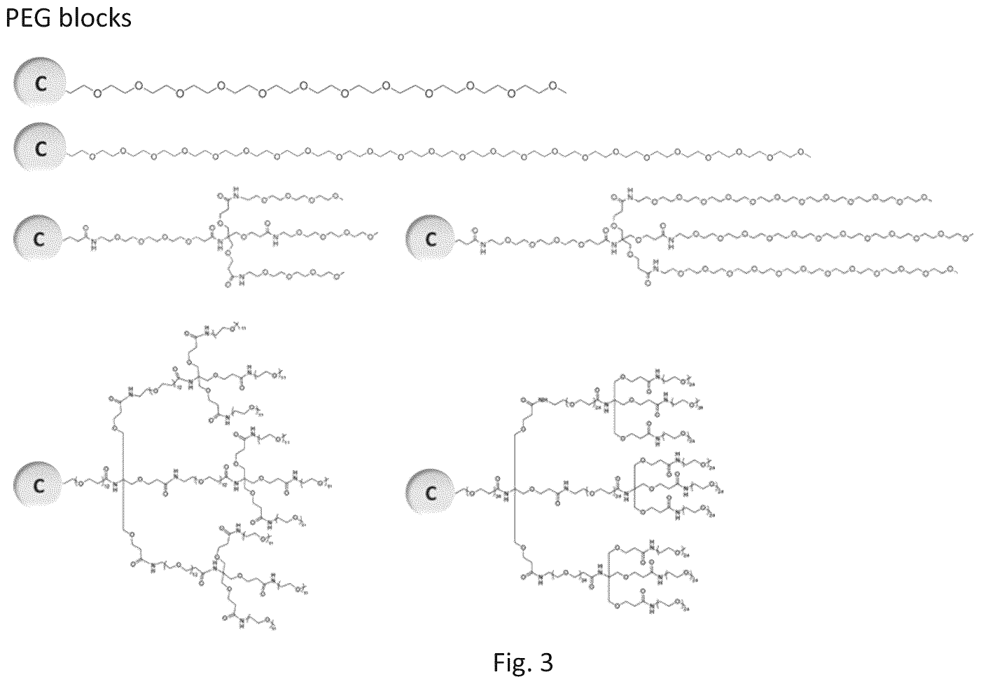

In some embodiments, non-limiting examples of PEG blocks may include those shown in FIG. 3. PEG blocks can be covalently linked to the translocating polymer or the incorporating nucleotide via one any of the couplings described herein.

### Peptide Blocks

Peptide blocks are macromolecular blocks designed to contain one or a series of derivative amino acid structures that can interact with a polymer sequence or residues comprising a nanopore. Peptide blocks for use in nanopore sequencing can be carefully designed to contain side chains that exhibit favorable interactions with the exposed residues of a nanopore, including MspA. For example, oppositely charged amino acid side chains interact via H-bonding and electrostatic forces to form salt bridges. Amino acids with polar side chains interact via hydrogen bonding and the bond strengths depend on the nature of H-donors and acceptors. Hydrophobic amino acids such as tyrosine, phenylalanine are also known to engage in OH-pi and CH—O type H-bonding. Peptide blocks for controlling translocation, controlling signal generation, and controlling enzymatic synthesis can be homogeneous in composition or contain a mixture of either naturally-occurring or synthetic amino acids. Methods by which control over the aforementioned aspects are achieved, are similar to those described for ONBs and PEG blocks. Geometry of peptide blocks can be modified to provide additional steric and conformational effects. For instance, increasing degree of branching in peptide blocks can increase their cross-sectional area while circularizing a linear sequence can provide a rigid block. In some embodiments, the peptide block includes a compound having one of the following structures:

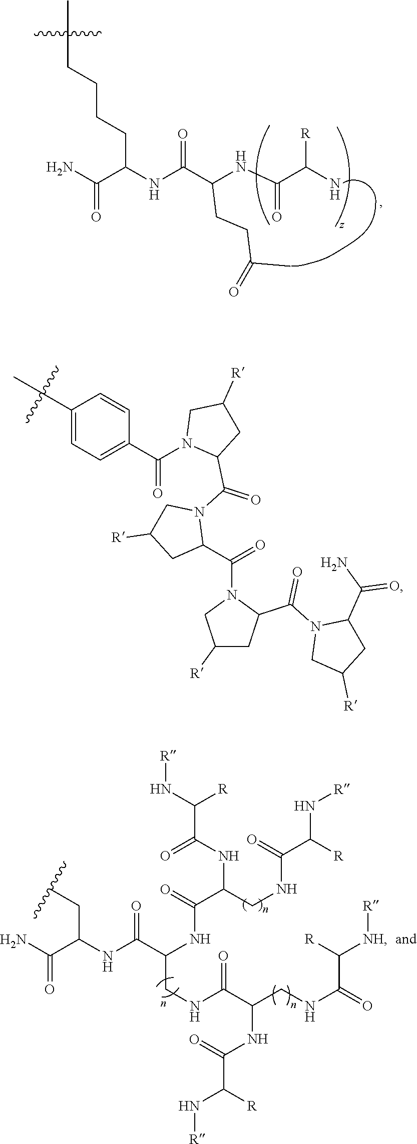

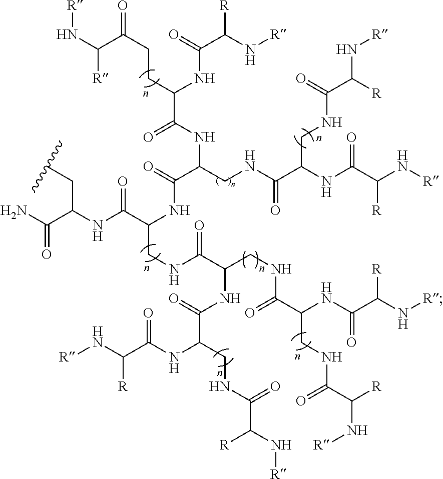

wherein each R is independently a side chain residue of an amino acid selected from the group consisting of

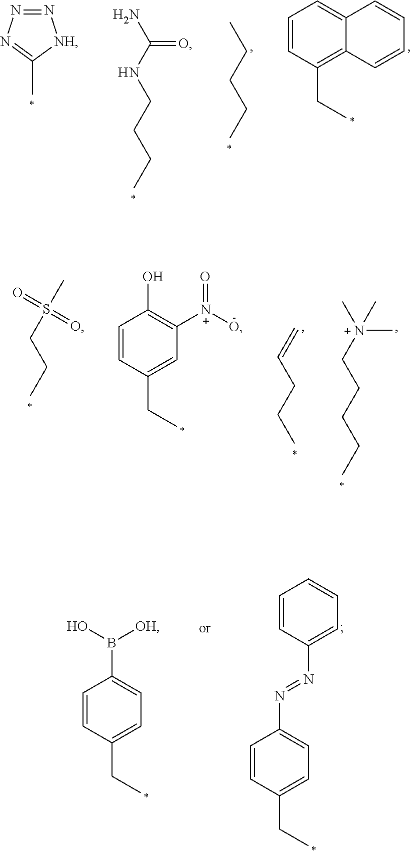

R′ is —OH, —CO₂⁻, —OSO₃⁻, or —OPO₃²⁻; R″ is H, acyl, acryl, alloc, benzoyl, BOC, Fmoc, formyl, or Cbz; z is an integer from 3-6; and n is an integer from 1-5.

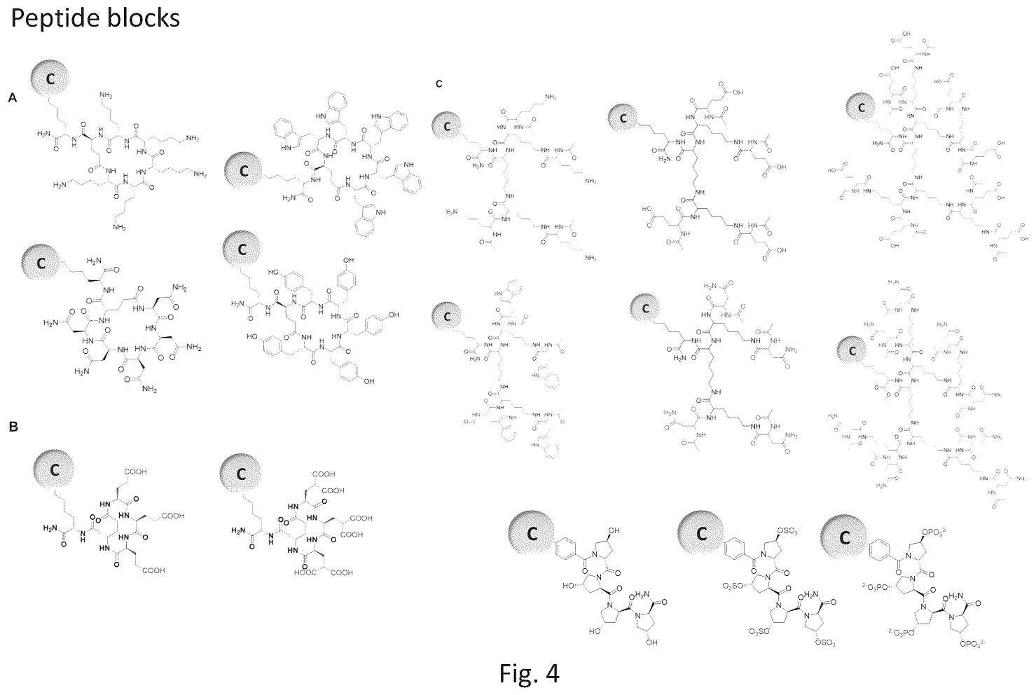

FIG. 4 shows non-limiting examples of peptide blocks. The peptide block(s) can be covalently linked to the translocating polymer or the incorporating nucleotide via one or any of the couplings described herein. Some non-limiting examples of peptide blocks are also reproduced below:

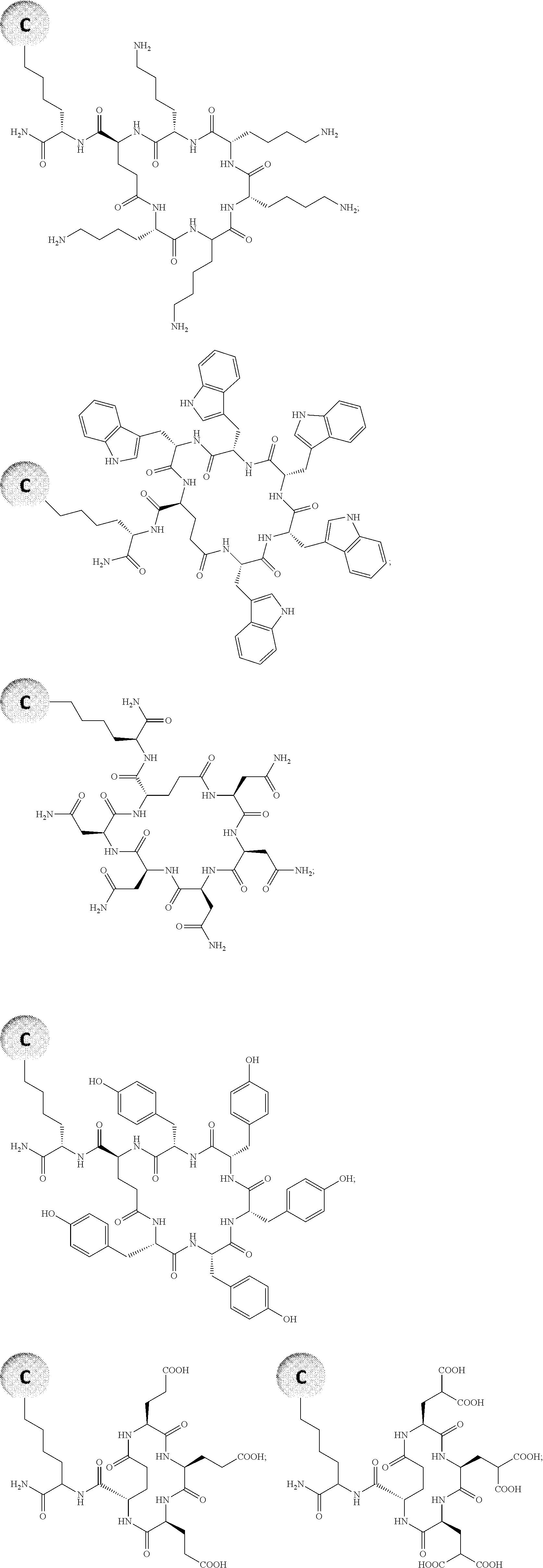

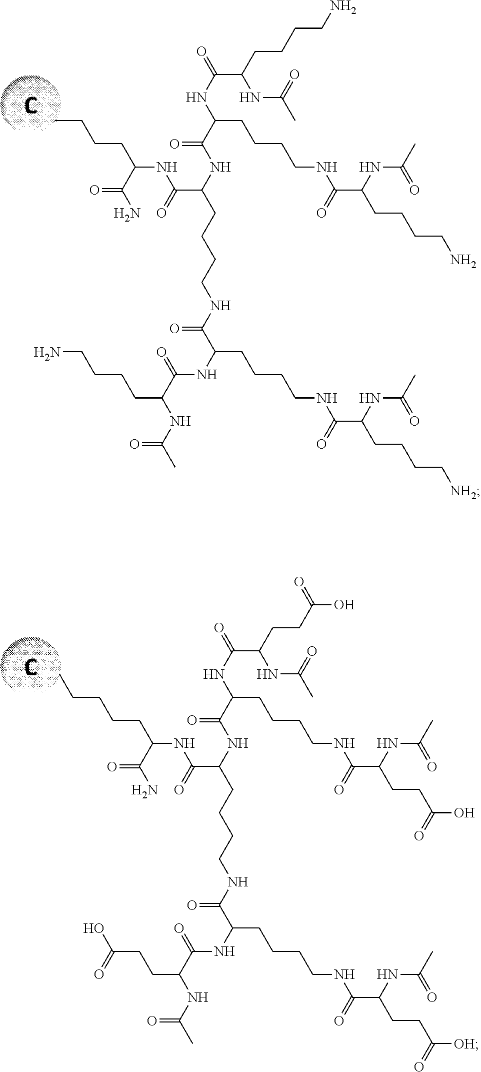 [SMILES: CCCCC(NC(=O)C(CCCCNC(=O)C(CCCCNC(=O)C(CCCCN)NC(C)=O)NC(=O)C(CCCCN)NC(C)=O)NC(=O)C(CCCCNC(=O)C(CCCCN)NC(C)=O)NC(=O)C(CCCCN)NC(C)=O)C(N)=O ; CCCCCC(NC(=O)C(CCCCNC(=O)C(CCCCNC(=O)C(CCC(=O)O)NC(C)=O)NC(=O)C(CCC(=O)O)NC(C)=O)NC(=O)C(CCCCNC(=O)C(CCC(=O)O)NC(C)=O)NC(=O)C(CCC(=O)O)NC(C)=O)C(N)=O]

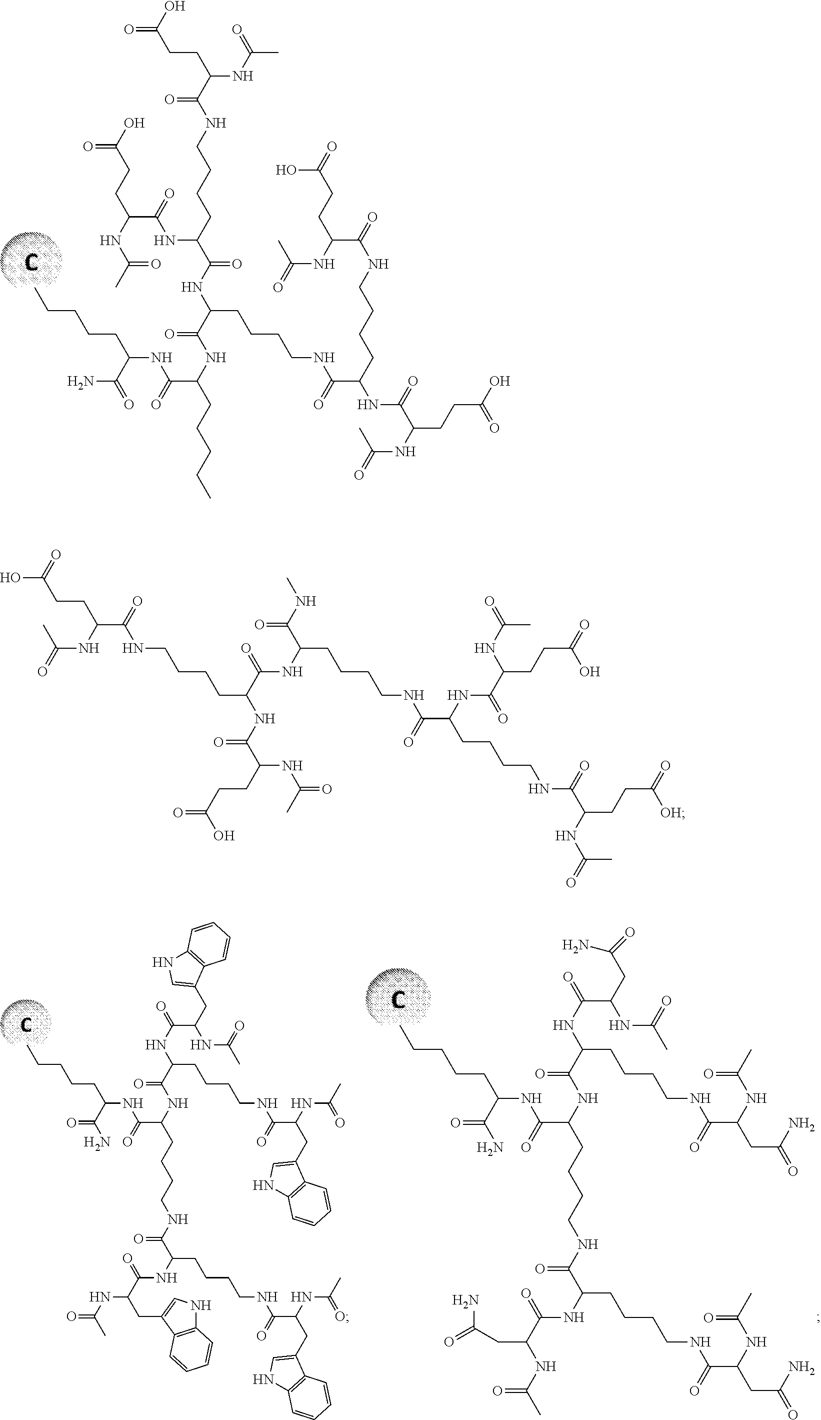

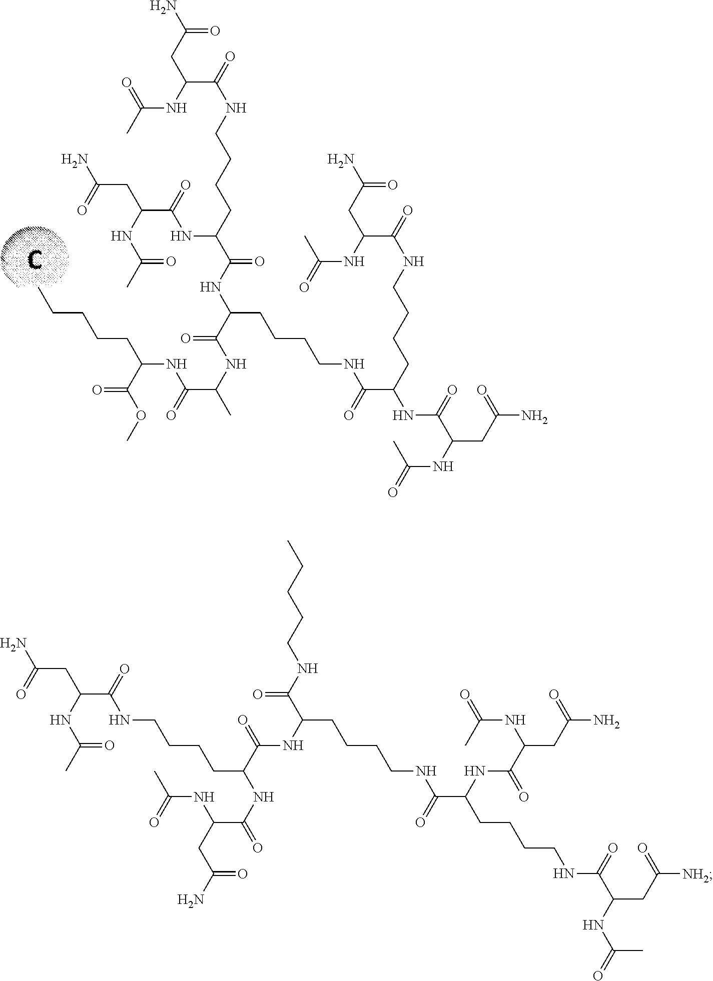 [SMILES: CCCCCC(NC(=O)C(C)NC(=O)C(CCCCNC(=O)C(CCCCNC(=O)C(CC(N)=O)NC(C)=O)NC(=O)C(CC(N)=O)NC(C)=O)NC(=O)C(CCCCNC(=O)C(CC(N)=O)NC(C)=O)NC(=O)C(CC(N)=O)NC(C)=O)C(=O)OC ; CCCCCNC(=O)C(CCCCNC(=O)C(CCCCNC(=O)C(CC(N)=O)NC(C)=O)NC(=O)C(CC(N)=O)NC(C)=O)NC(=O)C(CCCCNC(=O)C(CC(N)=O)NC(C)=O)NC(=O)C(CC(N)=O)NC(C)=O]

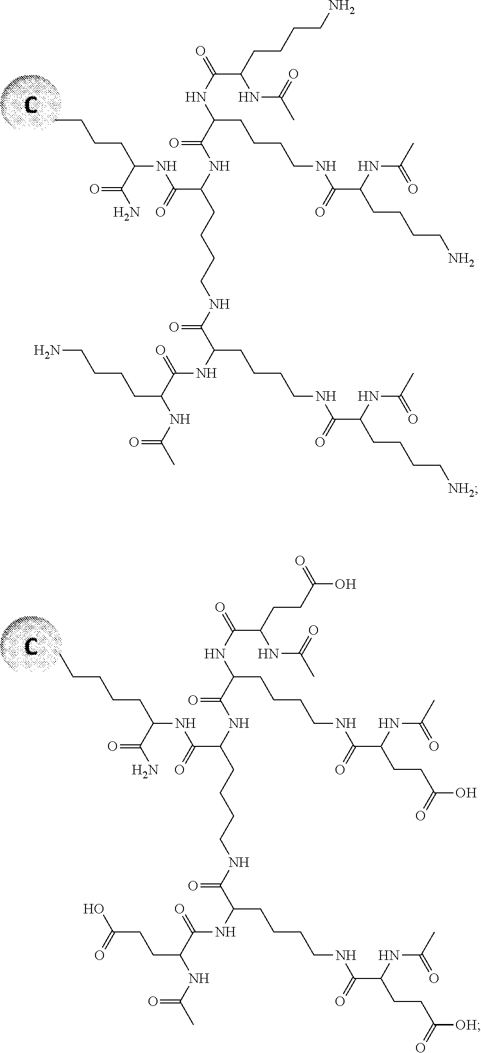 [SMILES: CCCCC(NC(=O)C(CCCCNC(=O)C(CCCCNC(=O)C(CCCCN)NC(C)=O)NC(=O)C(CCCCN)NC(C)=O)NC(=O)C(CCCCNC(=O)C(CCCCN)NC(C)=O)NC(=O)C(CCCCN)NC(C)=O)C(N)=O ; CCCCCC(NC(=O)C(CCCCNC(=O)C(CCCCNC(=O)C(CCC(=O)O)NC(C)=O)NC(=O)C(CCC(=O)O)NC(C)=O)NC(=O)C(CCCCNC(=O)C(CCC(=O)O)NC(C)=O)NC(=O)C(CCC(=O)O)NC(C)=O)C(N)=O]

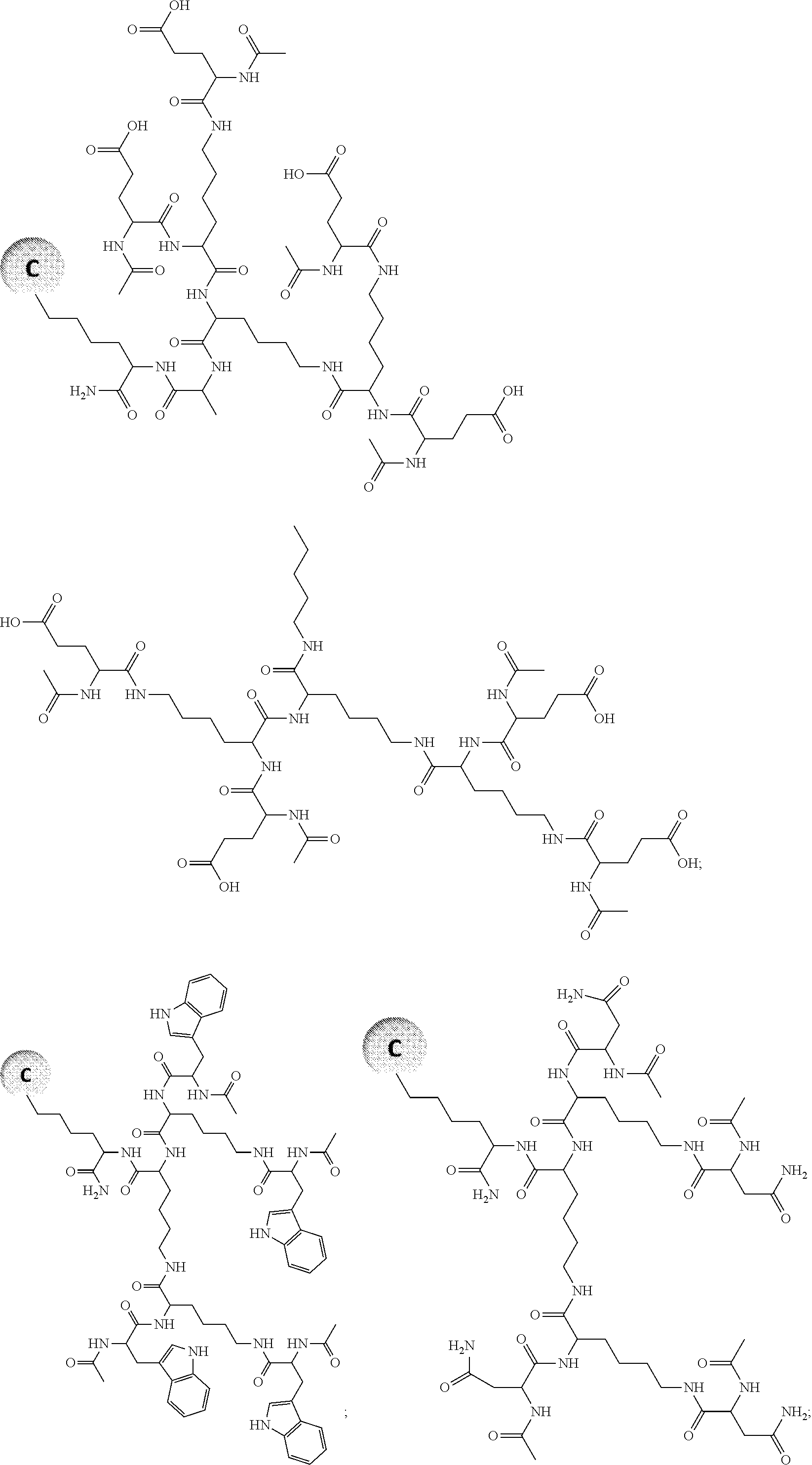

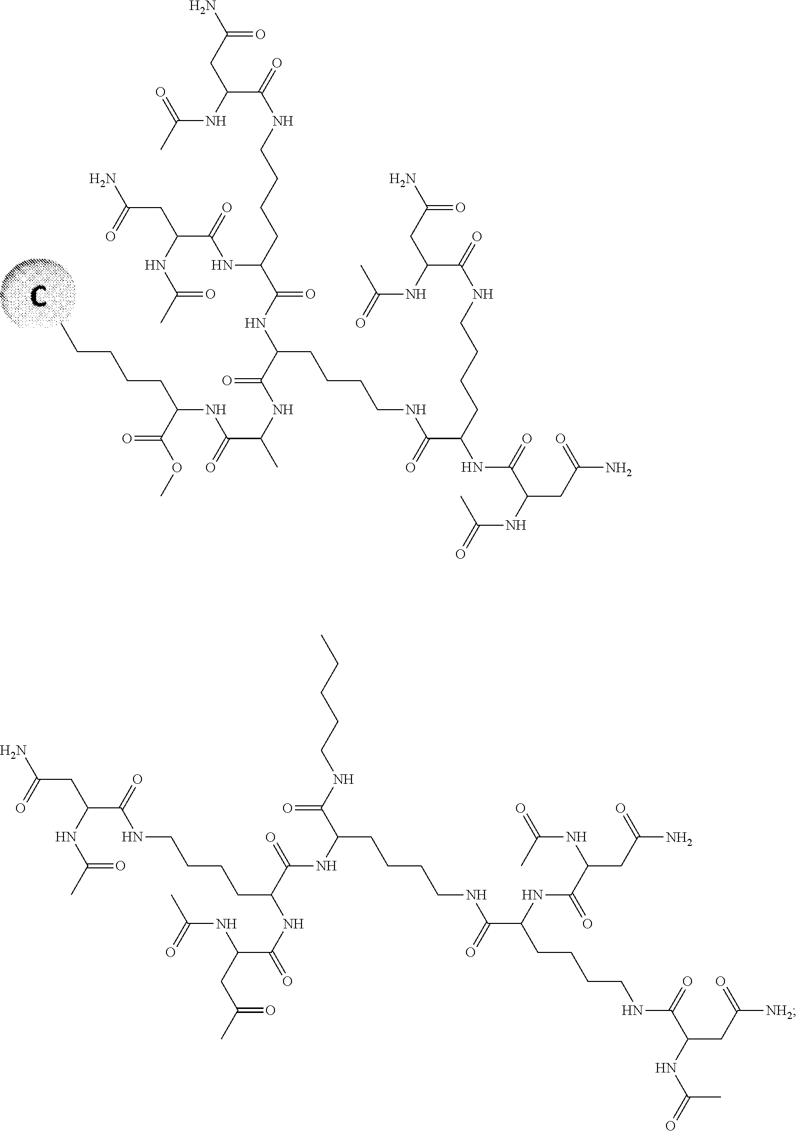 [SMILES: CCCCCC(NC(=O)C(C)NC(=O)C(CCCCNC(=O)C(CCCCNC(=O)C(CC(N)=O)NC(C)=O)NC(=O)C(CC(N)=O)NC(C)=O)NC(=O)C(CCCCNC(=O)C(CC(N)=O)NC(C)=O)NC(=O)C(CC(N)=O)NC(C)=O)C(=O)OC ; CCCCCNC(=O)C(CCCCNC(=O)C(CCCCNC(=O)C(CC(N)=O)NC(C)=O)NC(=O)C(CC(N)=O)NC(C)=O)NC(=O)C(CCCCNC(=O)C(CC(N)=O)NC(C)=O)NC(=O)C(CC(C)=O)NC(C)=O]

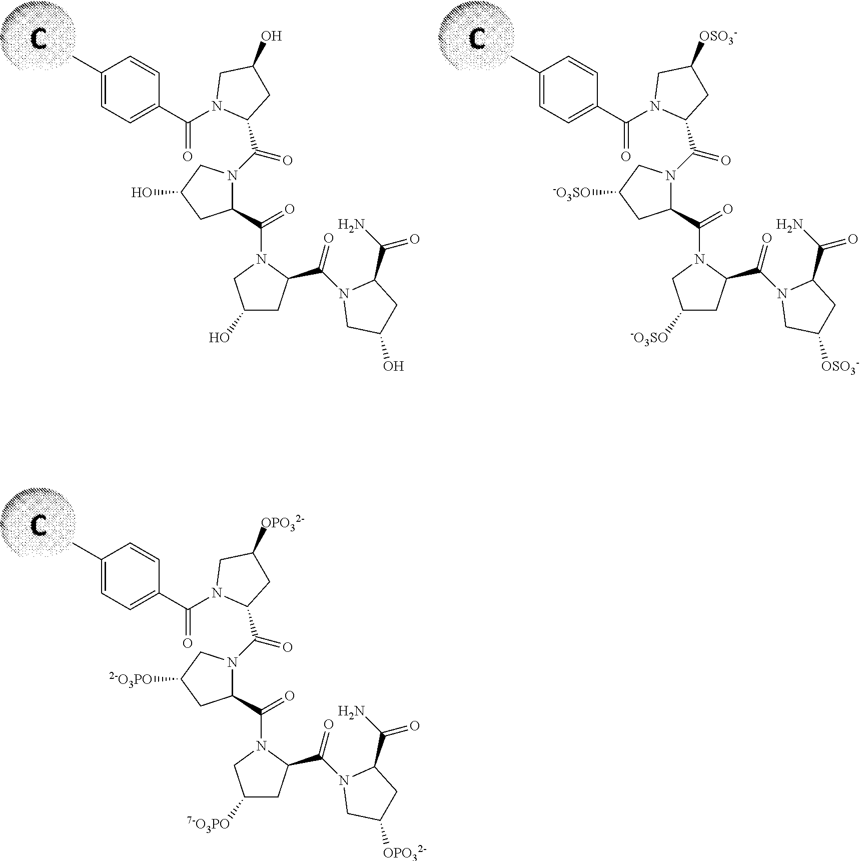

### Fluorinated Blocks

Fluorinated blocks are macromolecular blocks wherein fluorine atoms are incorporated into the constitutive chemical structure. Incorporation of fluorine atoms allows for fluorophilic domains, including those formed from a high density of amide bonds, to interact with the Fluorinated block. Fluorine may participate in orthogonal multipolar interactions with C═O groups, with further non-covalent interactions between F and H atoms. Molecular dynamic simulations have shown various short contacts between fluorine-containing groups, and domains inherent to protein structures. Fluorinated blocks as described herein can be designed to include covalent fluorine atoms or moieties to enable non-covalent interactions with the peptidic inner walls or the hydrophilic vestibule of a nanopore, including MspA. Perfluoroalkane blocks can interact via polar interactions with C═O groups of glutamate or aspartate and more fluorophilic guanidinium group of arginines. Similarly, fluorine interactions with sulfur atoms or cysteine residues in mutated MspA can potentially hold or slow down the translocating fluorinated motif within the pore. Other instances of fluorinated blocks include fluorinated peptides and as well as fluorinated oligonucleotides. Fluorinated block can include repeating difluorinated carbon units (—CF₂—) and repeating nucleotides with fluorinated groups.

The geometry of fluorinated blocks can be modified to provide additional sterics and conformational effects to provide more sustained fluorous contacts. For example, increasing the length or number of fluorine atoms within a linear block can increase their contact probability with residues in the pore while a branched fluorinated moiety with higher cross-sectional area has an added element of sterics that can serve as a hydrophobic rigid block. Inserting oxygen or peg units within the fluorinated block can provide various levels of hydrophilicity and flexibility to titrate its blocking capability. In some embodiments, fluorinated blocks can include compounds having one of the following structures:

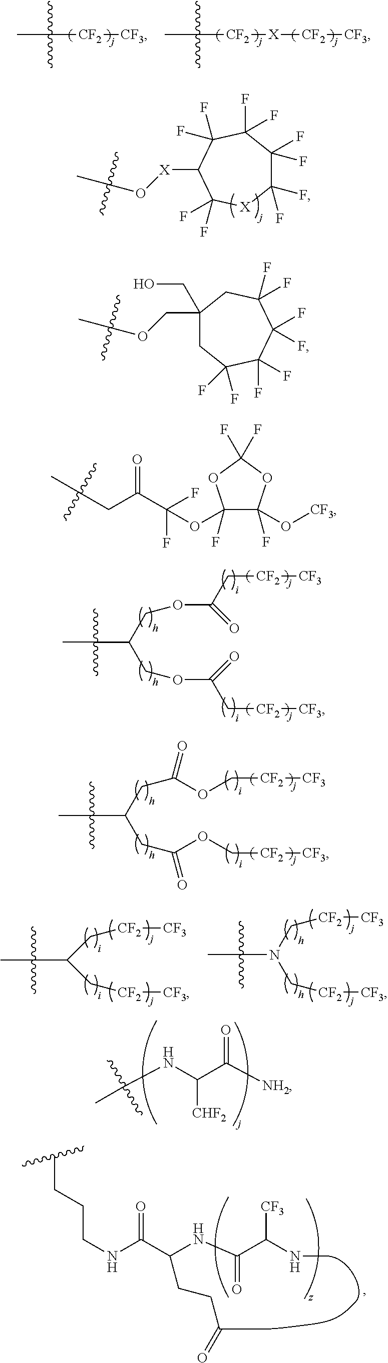

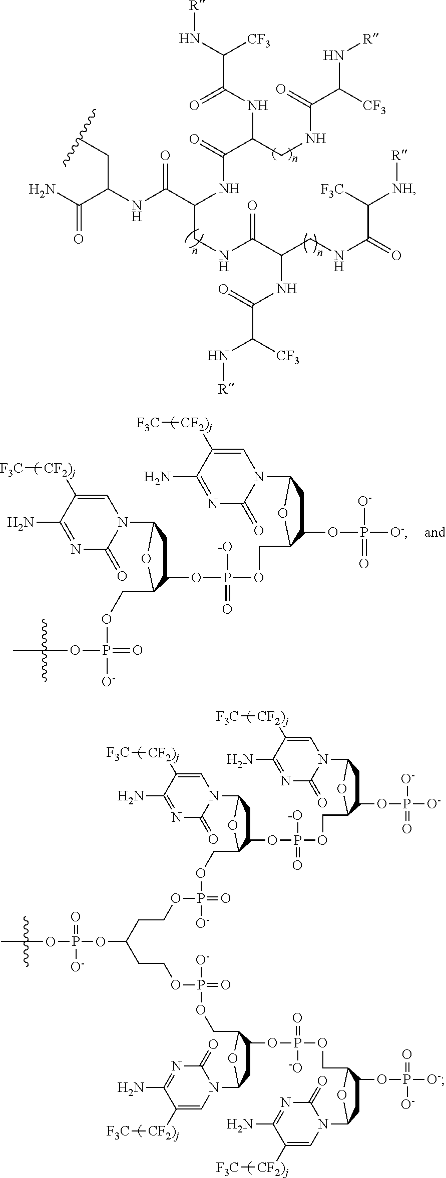

wherein h is an integer selected from 1-5, i is an integer selected from 1-5, j is an integer selected from 1-6; n is an integer selected from 1-5, z is an integer selected from 1-6; R″ is H, acyl, acryl, alloc, benzoyl, BOC, Fmoc, formyl, or Cbz; and X is C, O, PO₂, PO₃, or NR.

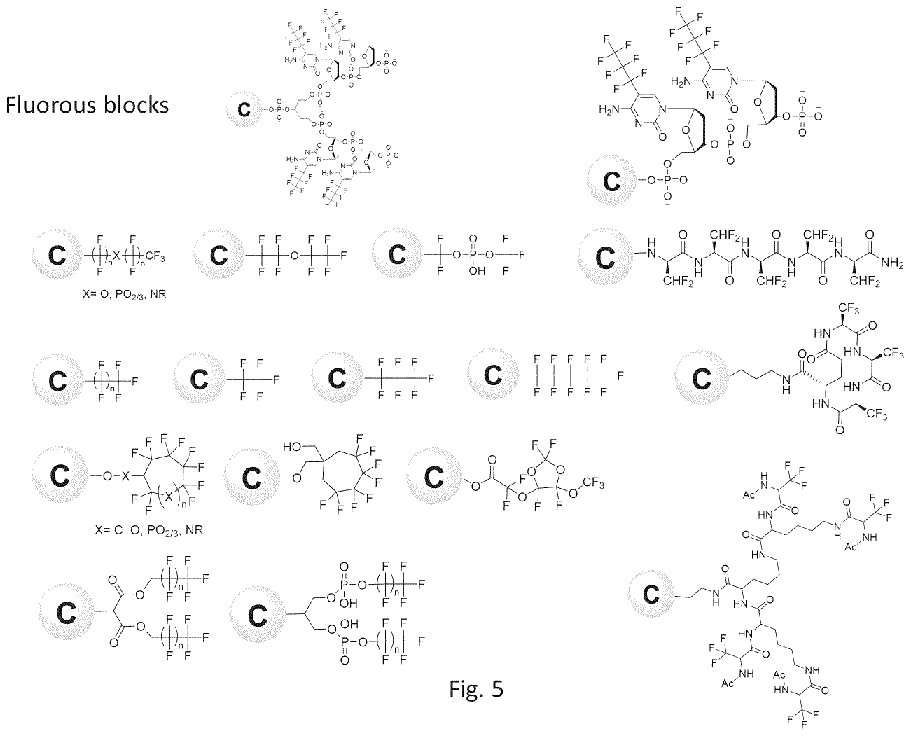

FIG. 5 discloses example fluorinated blocks, including perfluoroalkyls, hetero-atom bridged perfluoroalkyls, branched perfluoroalkyls, branched perfluoroalkyls, and fluoro-peptide blocks synthesized from fluoro-amino acid monomers, among others.

### EXAMPLES

### Example 1

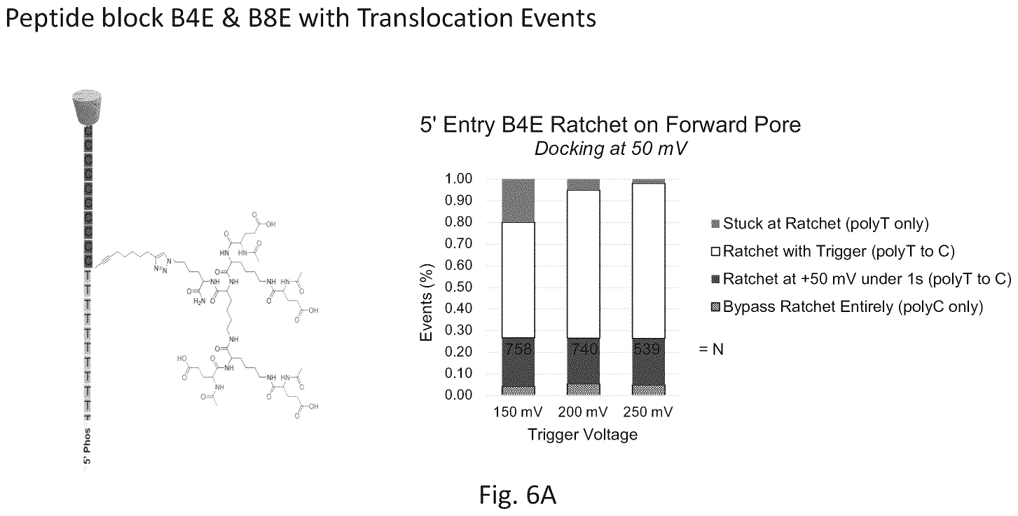

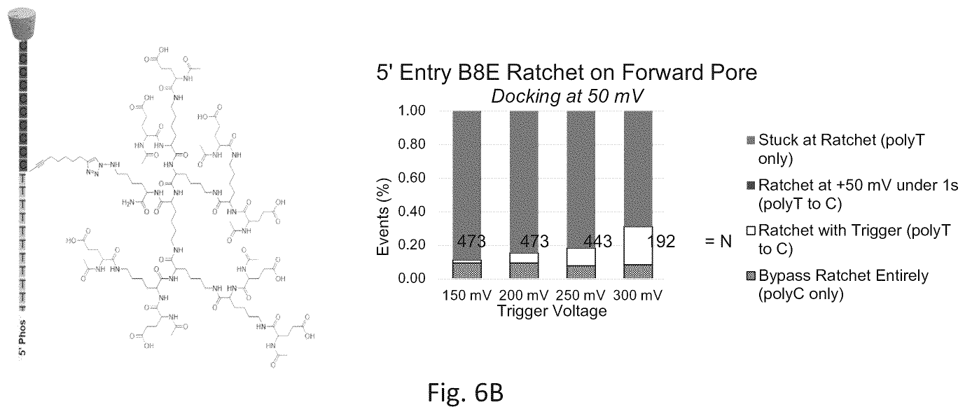

FIG. 6A illustrates the signal generation and attenuation of a polynucleotide attached to a macromolecular block, the macromolecular block (B4E) being a branched peptide with glutamate residues, with the attendant nanopore being MspA, to observe overall DNA translocation. The peptide block was conjugated to a synthetic oligo of a defined sequence and added to the cis side of a protein pore. A capture voltage of 50 mV was applied to attract the DNA into the constriction of the nanopore. After the block modified DNA was captured, a constant current that is characteristic of the DNA bases in the pore constriction was recorded. This current signature was used to determine the relative location (cis or trans) of the B4E peptide block in the nanopore. Subsequently, a transient pulse voltage of different magnitudes: 150 mV, 200 mV, or 250 mV was applied to drive the block through the pore, and translocation resumed until reaching the cap, a large protein (Neutravidin) that was attached to the terminal end of the translocation polymer which halted translocation.

As such a distribution of 4 types of events were observed when the DNA was captured in the pore (FIG. 6):
1. Stuck at Ratchet (PolyT only)—the current in the nanopore corresponds to polyT even after application of a transient pulse indicating block has blocked translocation of DNA.
2. Ratchet with trigger (PolyT to C)—the current in the nanopore after capturing the DNA corresponds to polyT and remains stable at this value. After application of a transient pulse the current signature changes to polyC indicating the peptide block has translocated through the pore.
3. Ratchet at +50 mV under 1s (polyT to C)—the current in the nanopore corresponds to polyT when the DNA is captured however it changes within 1 sec to a value corresponding to polyC indicating the block passes through the pore within 1 sec after being captured.
4. Bypass ratchet entirely (polyC only)—the current in the nanopore corresponds to polyC when the DNA is captured, indicating the block did not halt translocation and passed through the pore.

The distribution of events shows that at all trigger voltages, controlled translocation of DNA i.e., ‘Ratchet with trigger’ is the major event. Increasing the trigger voltage from 150 mV to 250 mV increases the controlled translocation rates from ˜55% to ˜73%, with a concomitant decrease in the number of “stuck at ratchet” events from ˜20% to ˜2%.

### Example 2

FIG. 6B further illustrates the signal generation and attenuation of a polynucleotide attached to another macromolecular block to observe overall DNA translocation. The macromolecular block (B8E), was a dendritic peptide with 8 glutamate end groups and the attendant nanopore was MspA. The peptide block was conjugated to a synthetic oligo of a defined sequence and added to the cis side of a protein pore. From the distribution of events, blocking of translocation (i.e. ‘Stuck at ratchet’) is the dominant translocation event across all trigger voltages. Increasing the trigger voltage from 150 mV to 300 mV resulted in increase of the controlled translocation events (Ratchet with trigger) from ˜3% to 36% with a concomitant decrease in blocking events. The higher efficiency of inhibiting translocation displayed by B8E compared to B4E can be attributed to its larger size and higher number of charged groups capable of non-covalent interactions.

### Example 3

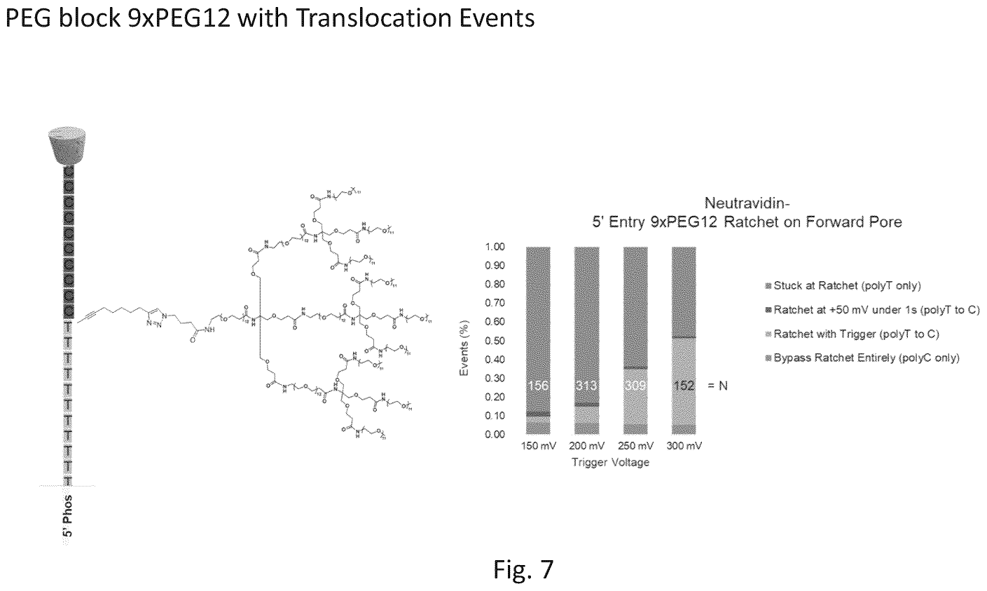

FIG. 7 further illustrates the signal generation and attenuation of a polynucleotide attached to another macromolecular block. A dendritic PEG block referred herein as 9xPEG12 was used to test its ability to control DNA translocation. At low trigger voltages, >80% of events correspond to complete blocking of translocation (i.e., ‘Stuck at PolyT’) while <5% of controlled translocation events are observed. Increasing the trigger voltage to 300 mV for 100 ms resulted in an increase of controlled translocation events to 45%.

### Example 4

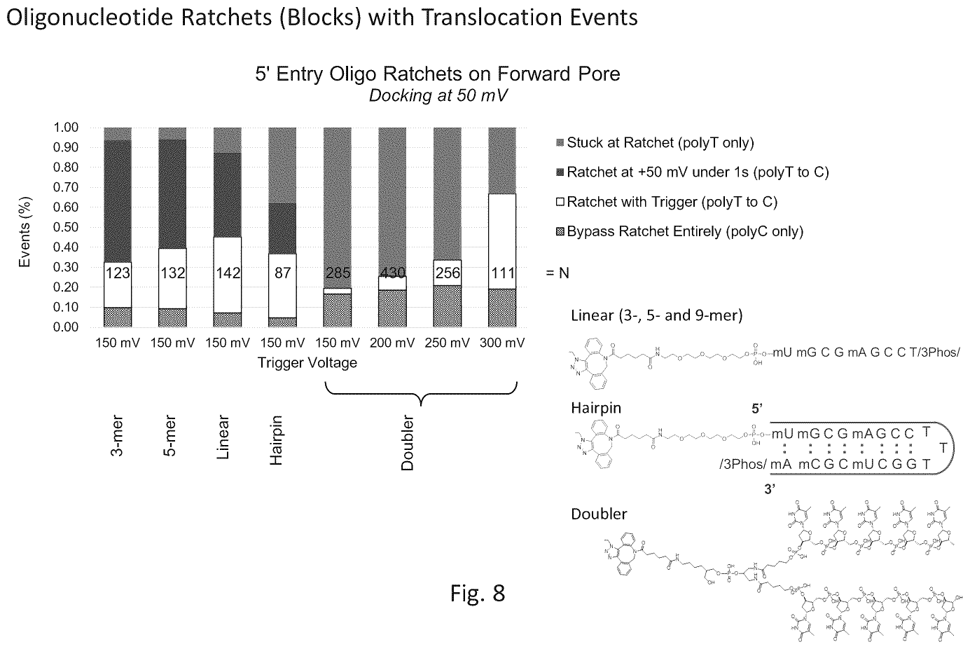

FIG. 8 further illustrates the signal generation and attenuation of a polynucleotide attached to another macromolecular block. A series of oligo ratchets (oligonucleotide blocks) comprising a linear structure (of either 3-, 5-, or 9-mer composition), a hairpin structure, or a doubler dendritic structure were measured for translocation events through MspA at varying voltages. Linear blocks showed 30-45% “Ratchet with Trigger (polyT to C)” with high levels of “Ratchet at +50 mV under 1s (polyT to C),” indicating the block passing through the power within 1 sec after being captured. Additionally, the hairpin structure showed 35-40% “Ratchet with Trigger (polyT to C)” activity at 150 mV. Multiple voltages were tested with the Doubler oligo block. Typically, at lower voltages, the rate of “Ratchet with Trigger (polyT to C)” ranged from ˜5% to 15% at or under 250 mV, which increased to ˜45% at 300 mV. Concurrently, the level of “Stuck at Ratchet (polyT only) events decreased as the voltage increased, from ˜80% to ˜30%.

### Example 5

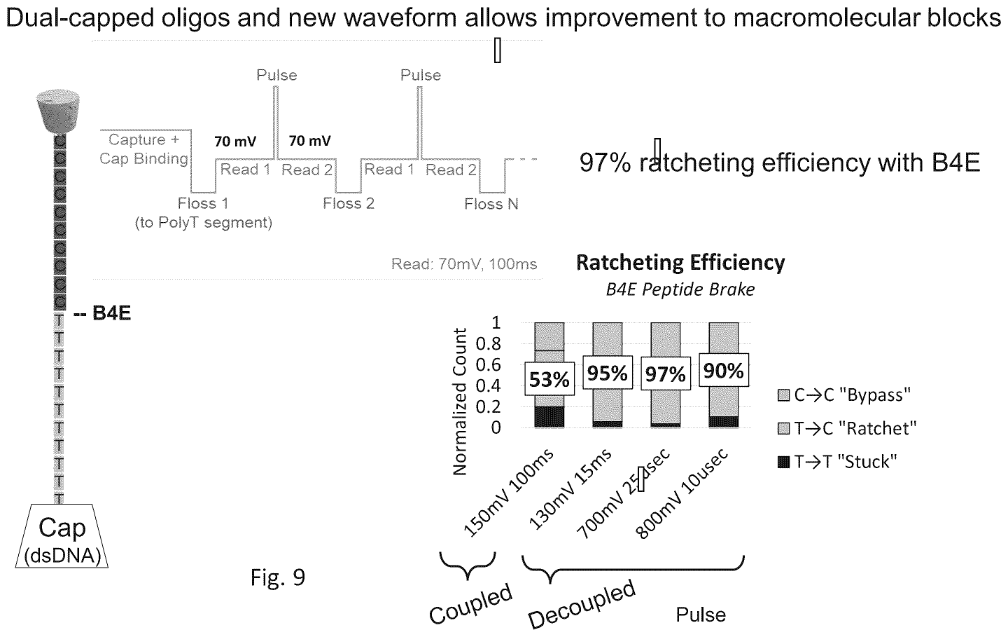

FIG. 9 illustrates the signal generation and attenuation of a polynucleotide attached to another macromolecular block. The polynucleotide was “dual capped” at the 5′ and 3′ end with constituent macromolecular caps to arrest or attenuate translocation. With this configuration a new waveform for signal generation provided increased efficiency of the constituent “ratchet” system. The macromolecular block (B4E) was tested attached to a polynucleotide with the aforementioned waveform generation scheme and dual caps. Overall ratcheting efficiency increased to 97% efficiency at higher voltages.

### Example 6

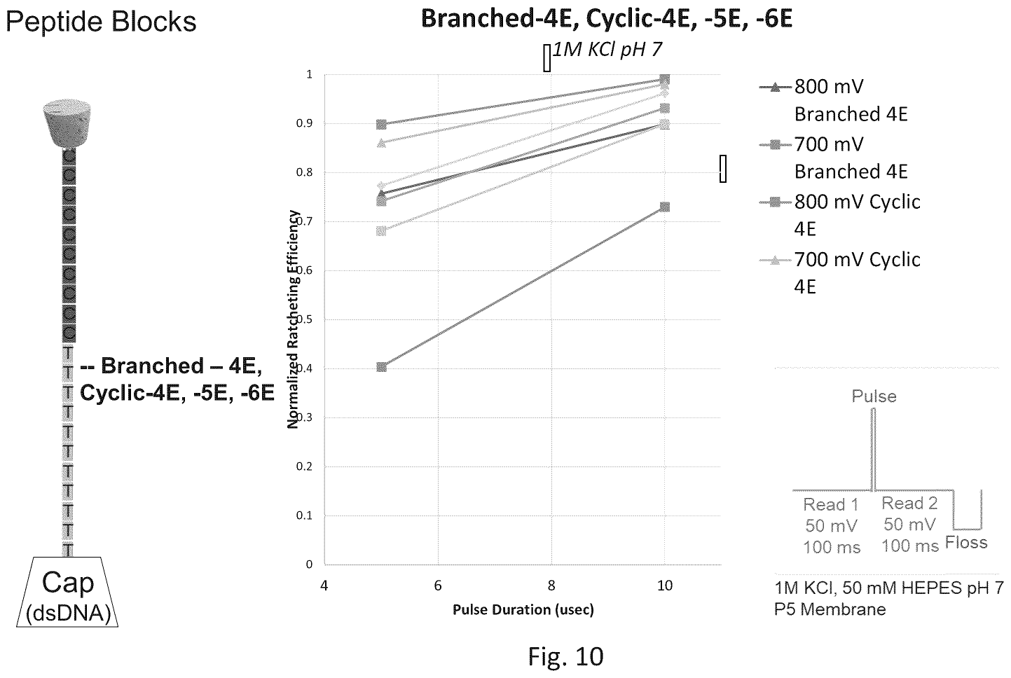

FIG. 10 illustrates the signal generation and attenuation of polynucleotides attached to macromolecular blocks. The polynucleotide was “dual capped” at the 5′ and 3′ end with constituent macromolecular caps to arrest or attenuate translocation. The macromolecular blocks were peptide blocks, comprising branched or cyclic-4E, -5E, or -6E structures. Overall ratcheting efficiency increased as a function of pulse duration, indicating tunable braking. Structures of the peptide blocks that were used are given below:

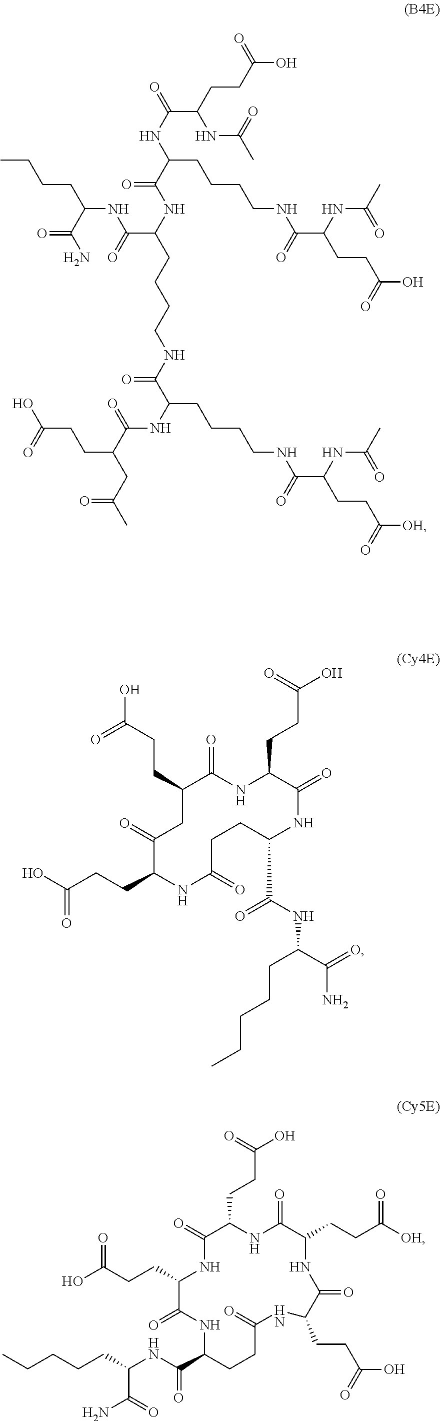

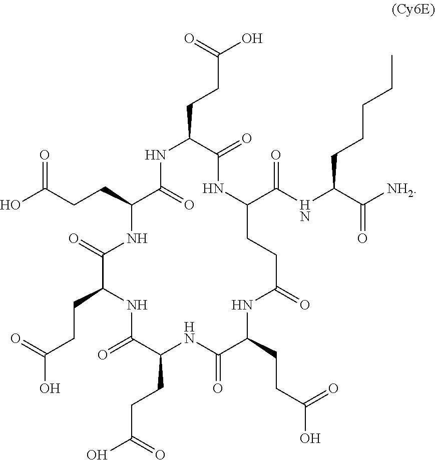 [SMILES: CCCCC[C@H](NC(=O)C1CCC(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N1)C(N)=O]

### Example 7

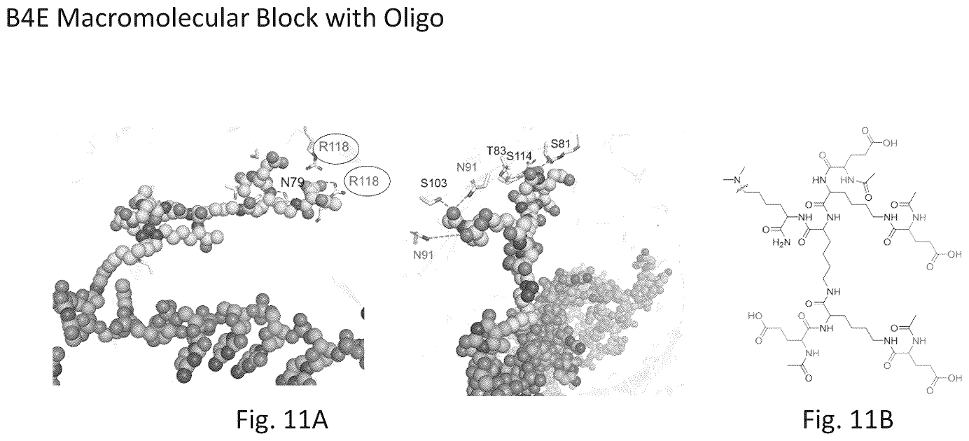

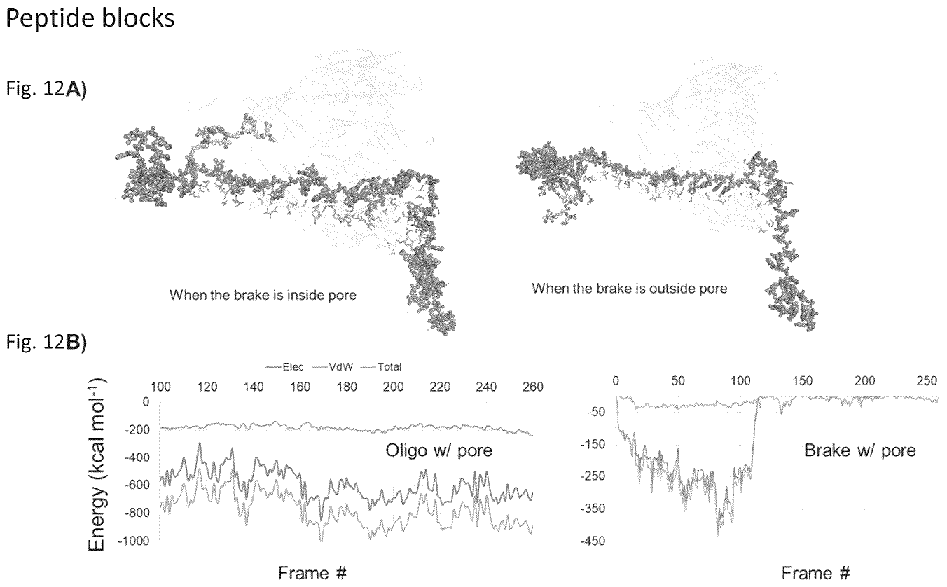

FIGS. 11A, 11B, 12A, and 12B illustrate molecular dynamic simulations showing the interaction of peptide blocks with MspA mutant (M2). The system was instantiated to translocating a DNA polymer, conjugated with the peptide block of interest, through MspA under a 700 mV field. The peptide block simulated was covalently attached to a translocating DNA polymer. In a 70 ns simulation, it was found that the glutamate side chains had sustained contact with R118 of MpA for ˜95% of the simulation time. The peptide block also exhibited other sustained contacts with N79, S103 and T83 which lasted for ˜50% of the simulation time (FIG. 11A). These observations partially elucidate the effectiveness of B4E in controlling DNA translocation in experiments.

In addition to B4E-pore interactions, the DNA polymer was also observed to have sustained interactions with the surface of the pore during simulations, even after the peptide block exits the pore constriction (FIG. 12A). Extracting the energetic contributions of the peptide block and DNA polymer generates an isolated energy graph, removing the effect of both components on translocation control. From FIG. 12B, it is apparent that in the case of B4E, the sum of electrostatic and Van-der-Waals interactions between DNA and pore are constant throughout the length of the simulation and exceeds that between B4E and pore.

### Example 8

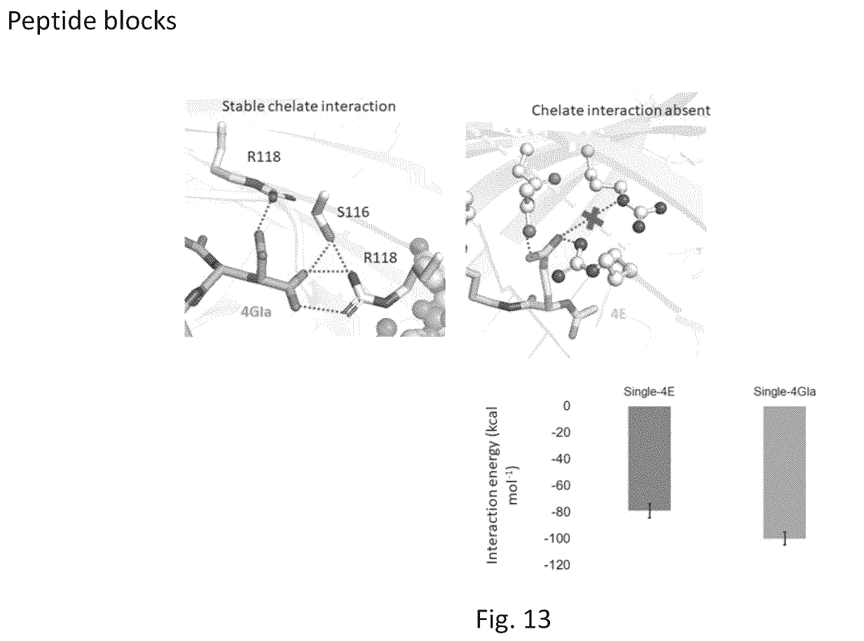

FIG. 13 illustrates molecular dynamic simulations showing the interaction of another peptide block with similar size as B4E but twice as many glutamate residues (referred here as B4Gla) with M2. The simulations show that the 4Gla interacts more strongly with MspA than B4E when the two adjacent carboxylates on 4Gla can form stable non-covalent interactions with arginine (R118) and serine (S116) residues.

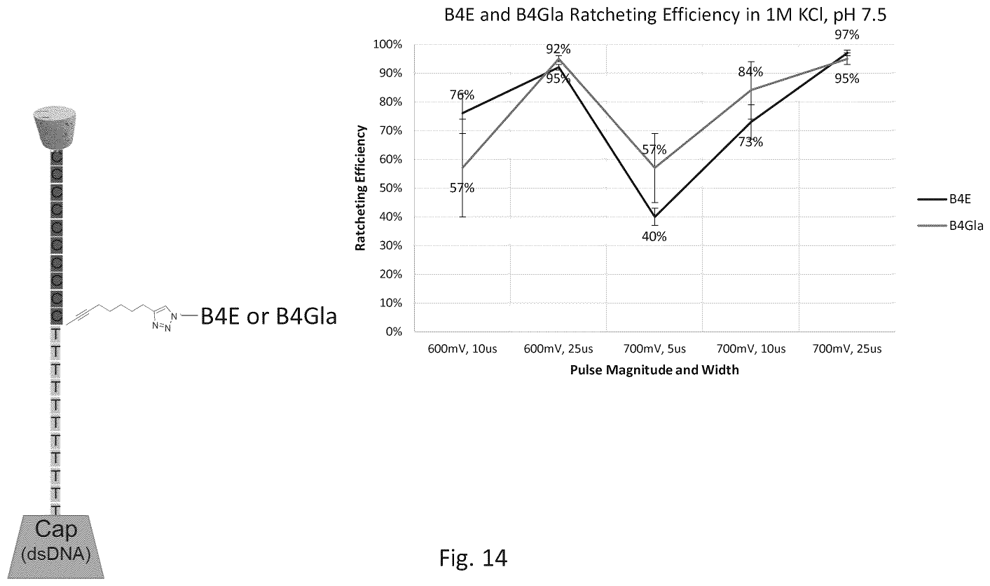

FIG. 14 illustrates experimental data sets of B4E and B4Gla and the ratcheting efficiency at various pulse magnitudes and widths. The experiment was performed at a pH of 7.5 in an aqueous solution of 1M KCl. The data demonstrates that B4E and B4Gla are able to interact with the pore sufficiently stably for a voltage pulse to translocate B4E and B4Gla through the pore. The graph in FIG. 14 shows that the ratcheting efficiency is generally the highest at 25 us pulse width, but the most efficient ratcheting for both structures was at 700 mV with 25 us pulse width. The lowest ratcheting efficiency is 700 mV and 5 us for B4E and the lowest ratcheting efficiency for B4Gla is at 600 mV and 10 us and 700 mV and 5 μs.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims. All references cited herein are incorporated by reference in their entirety.

The terminology used in the description presented herein is not intended to be interpreted in any limited or restrictive manner and unless otherwise indicated refers to the ordinary meaning as would be understood by one of ordinary skill in the art in view of the specification. Furthermore, embodiments may comprise, consist of, consist essentially of, several novel features, no single one of which is solely responsible for its desirable attributes or is believed to be essential to practicing the embodiments herein described. As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure is in the context of certain embodiments and examples, those of ordinary skill in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of ordinary skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for. one another in order to form varying modes or embodiments of the disclosure. Thus, it is intended that the scope of the present disclosure herein disclosed should not be limited by the particular disclosed embodiments described above.

What is claimed is:

1. A modified nucleotide comprising a modification covalently attached to its nucleobase or its sugar, wherein the modification comprises a macromolecule block selected from the group consisting of an oligonucleotide block, a polyethylene glycol block, a peptide block, and a fluorinated block.

2. The modified nucleotide of claim 1, wherein the macromolecule block is the oligonucleotide block, and the oligonucleotide block comprises natural DNA/RNA nucleotides, modified nucleic acids, or a combination thereof.

3. The modified nucleotide of claim 2, wherein the oligonucleotide block comprises a linear structure, a branched structure, a hairpin type structure, or a cyclic structure.

4. The modified nucleotide of claim 2, wherein the oligonucleotide block is selected from the group consisting of: 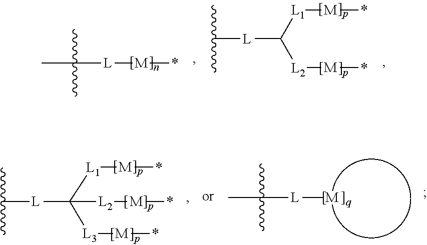
wherein each M is a natural nucleotide or a modified nucleic acid;
n is an integer selected from 1 to 70;
p is an integer selected from 1 to 30;
q is an integer selected from 2 to 10; and
* is an end group selected from the group consisting of 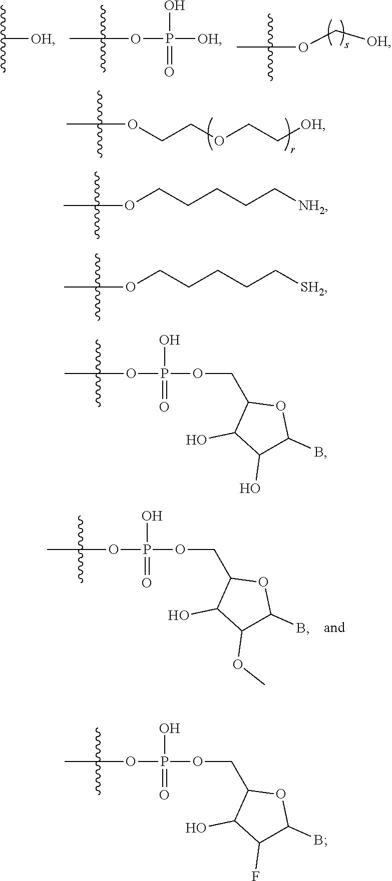
wherein r is 2, 3, 4, or 5, s is 3, 4, 5, 6, 7, 8, 9, 10, 11, or 12, and “B” is Uracil, Cytosine, Guanine, or Adenine.

5. The modified nucleotide of claim 1, wherein the macromolecule block is the polyethylene glycol block, and the polyethylene block comprises a linear or a branched structure.

6. The modified nucleotide of claim 5, wherein the polyethylene glycol block is selected from the group consisting of: 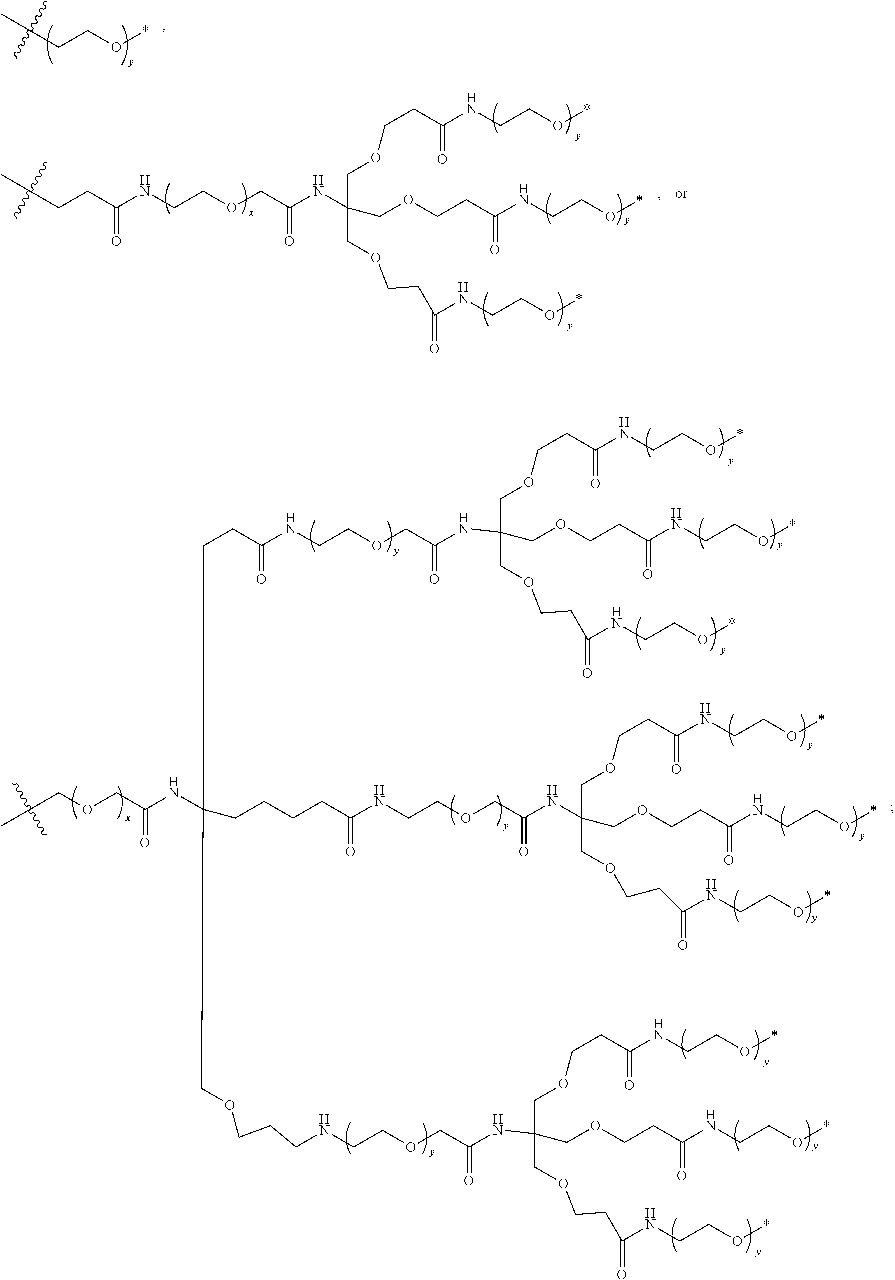
wherein x is an integer selected from 1 to 10;
y is an integer selected from 1 to 30; and
* is an end group selected from the group consisting of

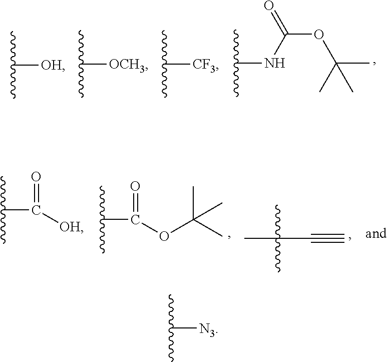

7. The modified nucleotide of claim 1, wherein the macromolecule block is the peptide block.

8. The modified nucleotide of claim 7, wherein the peptide block is selected from the group consisting of: 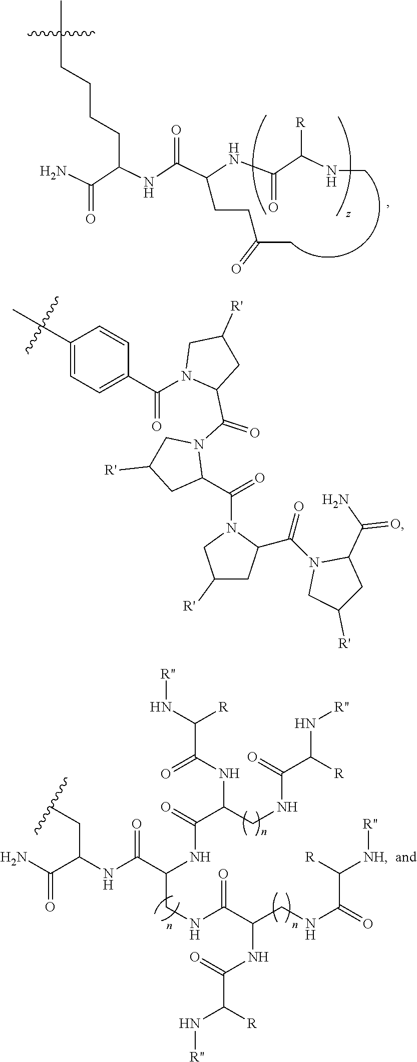 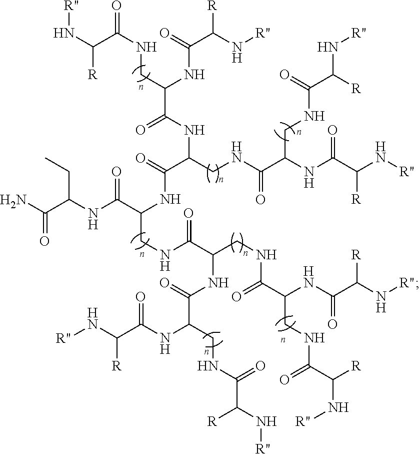
wherein each R is independently a side chain residue of an amino acid selected from the group consisting of 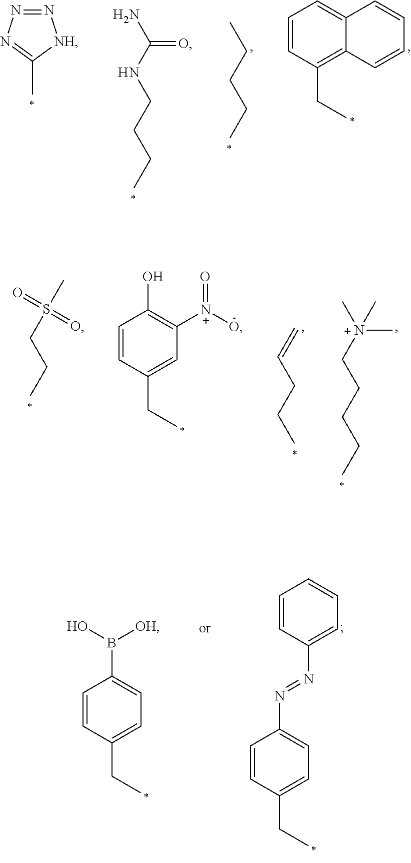
wherein R′ is —OH, —CO₂⁻, —OSO₃⁻, or —OPO₃²⁻;
R″ is H, acyl, acryl, alloc, benzoyl, BOC, Fmoc, formyl, or Cbz;
z is an integer selected from 3 to 6; and
n is an integer selected from 1 to 5.

9. The modified nucleotide of claim 1, wherein the macromolecule block is the fluorinated block.

10. The modified nucleotide of claim 9, wherein the fluorinated block comprises a perfluoroalkyl block, fluoro-peptide block, or perfluoroalkylated oligo block.

11. The modified nucleotide of claim 9, wherein the modification is selected from the group consisting of: 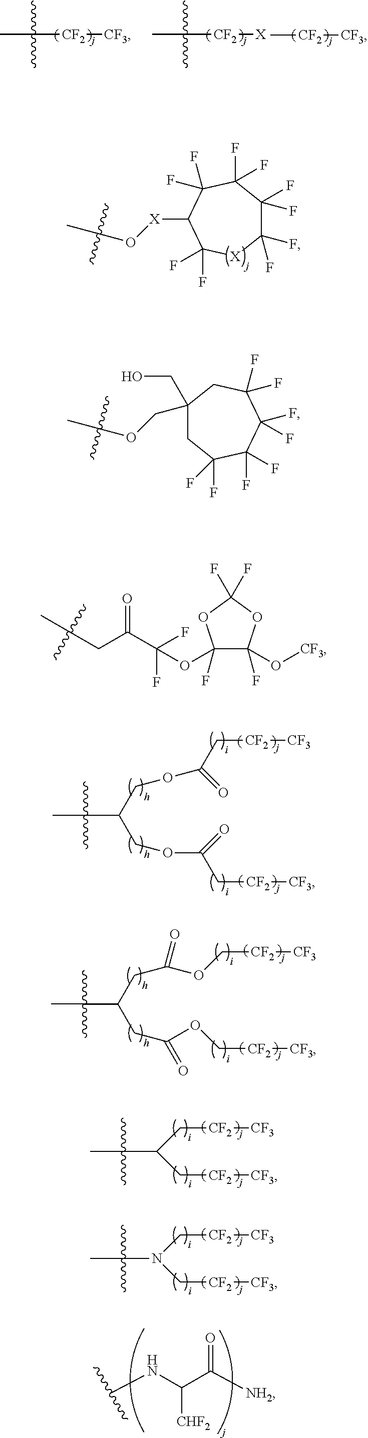 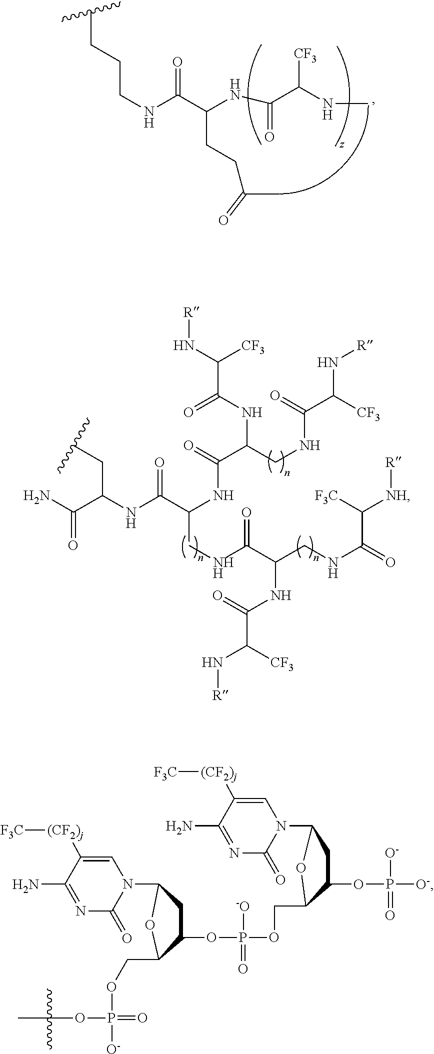
wherein:
h is an integer selected from 1 to 5;
i is an integer selected from 1 to 5;
j is an integer selected from 1 to 6;
n is an integer selected from 1 to 5;
z is an integer selected from 1 to 6;
R″ is H, acyl, acryl, alloc, benzoyl, BOC, Fmoc, formyl, or Cbz; and
X is C, O, PO₂, PO₃, or NR.

12. The modified nucleotide of claim 1, wherein the modification further comprises a covalent coupling between the macromolecule block and the modified nucleotide, wherein the covalent coupling comprises a moiety selected from the group consisting of amine-NHS ester, amine-imidoester, amine-pentofluorophenyl ester, amine-hydroxymethyl phosphine, carboxyl-carbodiimide, thiol-maleimide, thiol-haloacetyl, thiol-pyridyl disulfide, thiol-thiosulfonate, thiol-vinyl sulfone, aldehyde-hydrazide, aldehyde-alkoxyamine, hydroxy-isocyanate, azide-alkyne, azide-phosphine, transcyclooctene-tetrazine, norbornene-tetrazine, azide-cyclooctyne, and azide-norbornene.

13. The modified nucleotide of claim 1, wherein the modification further comprise one or more covalently linked moiety selected from the group consisting of dyes, synthetic polymers, and small molecules.

14. An oligonucleotide comprising one or more modified nucleotides according to claim 1.

15. A method for determining a sequence of a target polynucleotide in a nanopore-based sequencing system, the method comprising:
providing a daughter strand of the target polynucleotide comprising one or more modified nucleotides according to claim 1, and
reading the modified nucleotides by applying a reading voltage across a read head to identify a first reporter element in a constriction of a nanopore based on a first electrical response in the system, wherein one or more nucleotides translocate through a nanopore.

16. The method of claim 15, wherein the macromolecule block is configured to slow, pause, or halt the translocation.

17. A method for determining a sequence of a target polynucleotide in a nanopore-based sequencing system, the method comprising:
providing the target polynucleotide and a plurality of the macromolecule blocks according to claim 1 in an electrolyte for the nanopore-based sequencing system;
applying a voltage bias to cause the target polynucleotide to translocate through a constriction of a nanopore, wherein one or more of the plurality of the macromolecule blocks are non-covalently associated with the target polynucleotide; and
detecting and identifying one or more nucleotides as the nucleotides pass through the constriction based on an electrical response in the system.

18. The method of claim 17, wherein the macromolecule blocks are associated with the target polynucleotide via hydrogen bonding, Van-der-Waals interaction, ionic interaction, or hydrophobic interaction.

19. The method of claim 17, wherein the macromolecule blocks are configured to slow, pause, or halt the translocation.
